(19) United States
(12) Patent Application Publication
(10) Pub. No.: US 2010/0010078 A1
(43) Pub. Date: Jan. 14, 2010

(54) METHODS OF TREATING ANDROGEN DEPENDENT PROSTATE CANCER BY ADMINISTERING AN ACTIVE PHARMACEUTICAL INGREDIENT BEING FISETIN, 3,3',4',7-TETRAHYDROXYFLAVONE OR A DERIVATIVE THEREOF, IN AN ORAL, TRANSDERMAL OR TOPICAL DOSAGE FORM

(76) Inventors: Hasan Mukhtar, Fitchburg, WI (US); Naghma Khan, Madison, WI (US); Asim Mohammad, Bijnor (IN); Farrukh Afaq, Madison, WI (US)

Correspondence Address: QUARLES & BRADY LLP, 411 E. WISCONSIN AVENUE, SUITE 2040, MILWAUKEE, WI, 53202-4497, US

(21) Appl. No.: 12/412,945

(22) Filed: Mar. 27, 2009

Related U.S. Application Data
(60) Provisional application No. 61/040,303, filed on Mar. 28, 2008.

(51) Int. Cl. A61K 31/352; A61P 35/00

(52) U.S. Cl. 514/456

(57) ABSTRACT

Administration of fisetin (3,3′,4′,7-tetrahydroxyflavone) to a human male to treat androgen dependent prostate cancer. Fisetin is found in fruits and vegetables, such as strawberry, apple, persimmon, grape, onion and cucumber. Fisetin treatment, through modulations in cki-cyclin-cdk network, inhibits PI3K and Akt resulting in inhibition of cell growth followed by apoptosis of human prostate cancer LNCaP cells.

### CROSS REFERENCE TO RELATED APPLICATIONS

This application claims priority to and benefit of U.S. Provisional Patent Application Ser. No. 61/040,303 filed Mar. 28, 2008.

### STATEMENT CONCERNING GOVERNMENT INTEREST

This invention was made with United States government support awarded by the following agency: NIH CA120451. The United States government has certain rights in this invention.

### BACKGROUND OF THE INVENTION

Prostate cancer (PCa) is the most common cancer amongst men in the United States, and PCa is the second most common malignant cause of male death worldwide after lung cancer. (Jemal A et al., 2007, Cancer statistics, CA Cancer J. Clin. 57:43-66). The substantial mortality and morbidity associated with PCa (and its poor treatment options) have led a surge to develop novel means for its prevention. Diet-based agents for prevention and therapy are an attractive option in PCa for several reasons: incidence, prevalence, disease-related mortality, long duration of latency between premalignant lesions and clinically evident cancer and molecular pathogenesis, known hormonal influence on disease manifestations, and, epidemiologic data indicating that modifiable environmental factors may decrease risk. (Syed D N et al., 2007, Chemoprevention of prostate cancer through dietary agents: progress and promise, Cancer Epidemiol. Biomarkers Prev. 16:2193-2203; and, Adhami V M et al., 2006, Polyphenols from green tea and pomegranate for prevention of prostate cancer, Free Radic Res., 40:1095-1104).

Fisetin (3,3′,4′,7-tetrahydroxyflavone) is found in fruits and vegetables, such as strawberry, apple, persimmon, grape, onion and cucumber. (Arai Y et al., 2000, Dietary intakes of flavonols, flavones and isofiavones by Japanese women and the inverse correlation between quercetin intake and plasma LDL cholesterol concentration, J. Nutr. 130:2243-2250). Fisetin has been reported to inhibit cell-cycle progression in HT-29 human colon cancer cells. (Lu X et al., 2005, Fisetin inhibits the activities of cyclin-dependent kinases leading to cell cycle arrest in HT-29 human colon cancer cells. J. Nutr. 135:2884-2890). It has been shown to have antiproliferative effect on human PCa and breast cancer cell lines. (Haddad A Q et al., 2006, Novel antiproliferative flavonoids induce cell cycle arrest in human prostate cancer cell lines, Prostate Cancer Prostatic Dis. 9:68-76).

Fisetin has shown dose-dependent cytotoxic effects on SK-HEP-1 cells, accompanied by DNA fragmentation, induction of 32-kDa putative cysteine protease (CPP32) activity and increase of p53 protein. (Chen Y C et al., 2002, Wogonin and fisetin induction of apoptosis through activation of caspase 3 cascade and alternative expression of p21 protein in hepatocellular carcinoma cells SK-HEP-1, Arch. Toxicol. 76:351-359). It has also been reported that fisetin causes a decrease in intracellular peroxide level, activation of endonuclease and suppression of Mcl-1 proteins in the human leukemia cell line, HL-60. (Lee W R et al., 2002, Wogonin and fisetin induce apoptosis in human promyeloleukemic cells, accompanied by a decrease of reactive oxygen species, and activation of caspase 3 and Ca(2+)-dependent endonuclease, Biochem. Pharmacol. 63:225-236).

Fisetin activated the ERK (extracellular signal-regulated kinase) pathway, induced cAMP response element-binding protein (CREB) phosphorylation in rat hippocampal slices, and, enhanced object recognition in mice. (Maher P et al., 2006, Flavonoid fisetin promotes ERK-dependent long-term potentiation and enhances memory, Proc. Natl. Acad. Sci. USA. 103:16568-16573). Fisetin has been found to suppress tumor necrosis factor (TNF), various inflammatory agents, and carcinogen-induced nuclear factor kappa B (NF-κB) activation blocked the phosphorylation and degradation of IκBα by inhibiting IKKα activation and suppression of the phosphorylation and nuclear translocation of p65. Fisetin has also been found to suppress NF-κB-dependent reporter gene expression and NF-κB reporter activity induced by TNFR1, TRADD, TRAF2, NIK, and IKK. (Sung B et al., 2007, Fisetin, an Inhibitor of Cyclin-Dependent Kinase 6, Down-Regulates Nuclear Factor-{kappa}B-Regulated Cell Proliferation, Antiapoptotic and Metastatic Gene Products through the Suppression of TAK-1 and Receptor-Interacting Protein-Regulated I{kappa}B{alpha} Kinase Activation, Mol. Pharmacol. 71:1703-1714).

Recently, it was reported that fisetin (as well as quercetin, myricetin, quercetagetin,(-)-epigallocatechin gallate (EGCG), and theaflavins) markedly inhibited epidermal growth factor (EGF)-induced cell transformation of mouse epidermal JB6 Cl 41 cells. (Ichimatsu D et al., 2007, Miyamoto K. Structure-activity relationship of flavonoids for inhibition of epidermal growth factor-induced transformation of JB6 Cl 41 cells, Mol. Carcinog. 46:436-445).

Fisetin has further been found to induce phase II enzymes such as NADPH:quinone oxidoreductase (QR) activity in murine hepatoma IcIc7 cells. Transfection studies using a human QR antioxidant/electrophile-response element (ARE/EpRE) reporter construct demonstrated that fisetin activated the ARE/EpRE. (Hou D X et al., 2001, Fisetin induces transcription of NADPH:quinone oxidoreductase gene through an antioxidant responsive element-involved activation, Int. J Oncol. 18:1175-1179). Fisetin treatment of HL60 cells also caused high expression of NF-κB, activation of MAPK p38, an increase of phosphoprotein levels and inhibition of enzymes involved in redox status maintenance, (de Sousa R R et al., 2007, Phosphoprotein levels, MAPK activities and NFkappaB expression are affected by fisetin, J. Enzyme Inhib. Med. Chem. 22:439-444).

### SUMMARY OF THE INVENTION

One aspect of the invention is a method of treating androgen dependent prostate cancer in a human comprising the acts or steps of administering a therapeutically effective dose of an active pharmaceutical ingredient comprising fisetin according to the formula

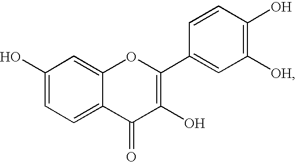

or, a prodrug, salt, hydrate, solvate or solute thereof.

Another aspect of the invention is a method of treating androgen dependent prostate cancer in a human comprising the steps or acts of orally administering a therapeutically effective dose of an active pharmaceutical ingredient comprising fisetin according to the formula

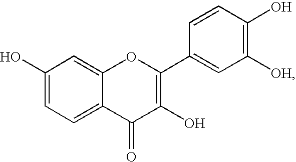

or, a prodrug, salt, hydrate, solvate or solute thereof.

Another aspect of the invention is a method of treating androgen dependent prostate cancer in a human comprising the steps or acts of transdermally administering a therapeutically effective dose of an active pharmaceutical ingredient comprising fisetin according to the formula

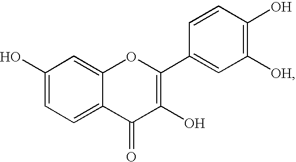

or, a prodrug, salt, hydrate, solvate or solute thereof.

Another aspect of the invention is a method of treating androgen dependent prostate cancer in a human comprising the steps or acts of topically administering a therapeutically effective dose of an active pharmaceutical ingredient comprising fisetin according to the formula

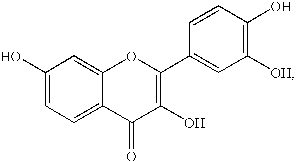

or, a prodrug, salt, hydrate, solvate or solute thereof.

In an exemplary embodiment of any of the above methods, the therapeutically

effective dose is around 50 mg/kg_{BW}.

### BRIEF DESCRIPTION OF DRAWINGS OF THE EXEMPLARY EMBODIMENTS

FIGS. 1, 2 and 3 demonstrate the effect of fisetin on cell-viability and interaction with the ligand binding domain (LBD) of androgen receptor (AR), whereby FIG. 1 shows the effect of fisetin on cell growth, whereby LNCaP, CWR22Ru1 and PrEC cells were treated with 10, 20, 40, and 60 μM of fisetin for 48 h and the viability of cells was determined by the MTT assay, whereby the data are expressed as the percentage of viable cells and represent the means ±SE of three experiments in which each treatment was performed in multiple wells (*P<0.001) compared with control (0 μM) group, whereby FIG. 2 shows the effect of fisetin on androgen agonist R1881-induced cell growth, whereby LNCaP cells were treated with R1881 (1 nM), Casodex (10⁻⁷M) and combination of R1881 (1 nM) and fisetin (10-60 μM) for 48 h and the viability of cells was determined by the 3-[4,5-dimethylthiazol-2-yl]-2,5-diphenyl tetrazoliumbromide (MTT) assay, whereby the data are expressed as the percentage of cell viability and represent the means ±SE of three experiments in which each treatment was performed in multiple wells (*P<0.001) compared with control (0 μM) (§P<0.001 compared with R1881), and, whereby FIG. 3 shows that fisetin competes with a labeled AR ligand and physically interacts with its LBD. Results were analyzed using a two-tailed Student's t-test to assess statistical significance and P values <0.05 were considered significant.

FIGS. 4-6 demonstrate the effect of fisetin on protein and mRNA expression of AR (effect on its promotor activity) and interference of fisetin with N-C terminus interaction of AR, whereby FIG. 4 shows the effect of fisetin on protein expression (upper panel) and mRNA expression (lower panel) of AR as determined by western blot and RT-PCR analysis respectively in LNCaP cells, whereby (for westm blot analysis) total cell lysates were prepared and 40 μg protein was subjected to SDS-PAGE followed by immunoblot analysis and chemiluminescence detection, whereby equal loading of protein was confirmed by stripping the immunoblot and reprobing it for β-actin, whereby the immunoblots shown are representative of 3 independent experiments with similar results, whereby the values above the figures represent relative density of the bands normalized to β-actin, whereby in FIG. 5 LNCaP cells were transiently transfected with AR promoter reporter (pLARS) along with Renilla-Luc and treated with solvent (DMSO) and fisetin (10-60 μM) for 48 h, whereby the luciferase activity of the samples was measured and normalized to renilla luciferase, whereby the bar diagram represents the average of relative luciferase values of triplicate with means +SE, whereby FIG. 6 is a bar graph showing the results of an AR N-C terminus interaction assay performed in CV1 cells, whereby, 8 h post transfection, cells were washed, fresh media replaced and treated with R1881 (1 nM) and/or Casodex (10-7M) along with 10 and 20 μM of fisetin for 48 h after which cells were lysed and luciferase activity was measured, and whereby, the graphs represent the fold hormone induction compared to no hormone treated group which was set as 1.

FIGS. 7-10 demonstrate the effect of fisetin on the expression of androgen target gene PSA expression (protein and mRNA expression of PSA and AR-regulated reporter genes), whereby FIG. 7 shows the effect of fisetin on protein expression (upper panel) and mRNA expression (lower panel) of PSA as determined by RT-PCR in LNCaP cells, whereby total cell lysates were prepared and 40 μg protein was subjected to SDS-PAGE followed by immunoblot analysis and chemiluminescence detection, whereby equal loading of protein was confirmed by stripping the immunoblot and reprobing it for β-actin, whereby the immunoblots shown are representative of 3 independent experiments with similar results, whereby the values above the figures represent relative density of the bands normalized to β-actin, whereby FIG. 8 shows the effect of fisetin on secreted levels of PSA in LNCaP cells, whereby the cells were treated with fisetin (10-60 μM) for 48 h and then harvested, whereby the PSA levels were determined by enzyme linked immunoabsorbent assay (ELISA), whereby the data includes three experiments each conducted in duplicate, error bars represent mean +SE (*P<0.001 compared with control 0 μM group), whereby FIG. 9 involves LNCaP cells transiently transfected with PSA-Luc, whereby FIG. 10 involves LNCaP cells transiently transfected with MMTV-Luc, whereby reporters were treated 48 h post-transfection with or without 1 nM R1881 and with fisetin (10-60 μM), and, whereby the graphs represent the fold hormone induction compared to no hormone treated group which was set as 1. Results were analyzed using a two-tailed Student's t-test to assess statistical significance and P values <0.05 were considered significant.

FIGS. 11-12 demonstrate the effect of fisetin on DHT-stimulated protein expression of AR (FIG. 11) and PSA (FIG. 12) in LNCaP cells, whereby the cells were treated with 40 μM of fisetin for 48 h and then harvested, whereby cells were grown in 10% FBS, charcoal stripped fetal bovine serum (FBS) with 10, 50 and 100 nM of DHT with or without fisetin (40 μM) for 48 h, whereby total cell lysates were prepared and 40 μg protein was subjected to SDS-PAGE followed by immunoblot analysis and chemiluminescence detection, whereby equal loading of protein was confirmed by stripping the immunoblot and reprobing it for β-actin, whereby the immunoblots shown here are representative of 3 independent experiments with similar results, and, whereby the values above the figures represent relative density of the bands normalized to β-actin.

FIGS. 13-18 demonstrate the effect of fisetin on induction of apoptosis, whereby LNCaP cells were treated with fisetin (10 and 20 μM) and/or Casodex (10-7M) for 48 h, whereby the fluorescence was measured by a Zeiss 410 confocal microscope, and, whereby the data shown are from one representative experiment repeated two times with similar results, magnification ×400.

FIGS. 19-21 demonstrate the effect of fisetin on PCa CWR22Ru1 tumor growth and PSA secretion in athymic nude mice, whereby 24 animals were randomly divided into 8 animals in group 1 and 16 animals in group 2, whereby approximately one million CWR22Ru1 cells were subcutaneously injected in each flank of mouse to initiate tumor growth, whereby 24 h after cell implantation, 8 animals of the first group of animals received intraparatoneal (i.p.) injection of DMSO (30 μl) and served as control, whereby the 16 animals of group 2 received i.p. injection of fisetin (1 mg/animal) in 30 μl of DMSO twice weekly, whereby once tumors started to grow, their sizes were measured twice weekly and the tumor volume was calculated, whereby FIG. 19 is a graph showing average tumor volume of control group and fisetin treated mice plotted over 45 days after tumor cell inoculation, whereby values represent mean ±SD of 16 tumors in eight animals (*P<0.001 vs. the control group of mice), whereby FIG. 20 shows the number of mice remaining with tumor volumes of 1,200 mm³ after they received treatment with fisetin for the indicated days, whereby FIG. 21 is a graph showing serum PSA levels analyzed by ELISA, and, whereby values represent mean +SE of 8 animals (*P<0.001 vs. control group of mice). Results were analyzed using a two-tailed Student's t-test to assess statistical significance and P values <0.05 were considered significant.

FIG. 22 is a pictoral summary of a theoretical mechanism of fisetin action in prostate cancer cells, whereby fisetin decreases AR promoter activity leading to decrease in its expression, whereby fisetin competes with natural AR agonist DHT to physically interact with the expressed AR protein, whereby once bound, fisetin decreases the interdomain N/C interaction of AR leading to (1) a decrease in its stabilization and (2) a decrease in AR transactivation function, and, whereby the resultant decrease in expression of AR target genes occurs negatively influencing the growth of PCa cells in vitro and in vivo.

FIGS. 23 and 24 are graphs showing the treatment effect of fisetin on cell growth, whereby LNCaP, CWR22Ru1, PC-3 and PrEC cells were treated with fisetin (10-60 μM) for 24 h and 48 h, whereby the viability of cells was determined by the MTT assay, and, whereby the data are expressed as the percentage of cell viability and represent the means ±SE of three experiments in which each treatment was performed in multiple wells.

FIGS. 25-29 are graphs showing the effect of fisetin treatment on cell cycle distribution in LNCaP cells, whereby the cells treated with fisetin (10-60 μM; 48 h) were collected and stained with PI by using an apoptosis APO-DIRECT kit obtained form Phoenix Flow systems as per vendor's protocol followed by flow cytometry, whereby following FACS analysis cellular DNA histograms were further analyzed by ModfitLT V3.0, and, whereby the data are representative example for duplicate tests.

FIG. 30 shows the effect of fisetin treatment on protein expression of cyclin D1, cyclin D2 and cyclin E in LNCaP cells, whereby the cells were treated with fisetin (10-60 μM; 48 h) and then harvested, whereby total cell lysates were prepared and 40 μg protein was subjected to SDS-PAGE followed by immunoblot analysis and chemiluminescence detection, whereby equal loading of protein was confirmed by stripping the immunoblot and reprobing it for β-actin, whereby the immunoblots shown here are representative of three independent experiments with similar results, and, whereby the values above the figures represent relative density of the bands normalized to β-actin.

FIG. 31 shows the effect of fisetin treatment on protein expression of cdk2, cdk4 and cdk6 in LNCaP cells, whereby the cells were treated with fisetin (10-60 μM; 48 h) and then harvested, whereby total cell lysates were prepared and 40 μg protein was subjected to SDS-PAGE followed by immunoblot analysis and chemiluminescence detection, whereby equal loading of protein was confirmed by stripping the immunoblot and reprobing it for β-actin, whereby the immunoblots shown here are representative of three independent experiments with similar results, and, whereby the values above the figures represent relative density of the bands normalized to β-actin.

FIG. 32 shows the effect of fisetin treatment on protein expression of WAF1/p21 and KIP1/p27 in LNCaP cells, whereby the cells were treated with fisetin (10-60 μM; 48 h) and then harvested, whereby total cell lysates were prepared and 40 μg protein was subjected to SDS-PAGE followed by immunoblot analysis and chemiluminescence detection, whereby equal loading of protein was confirmed by stripping the immunoblot and reprobing it for β-actin, whereby the immunoblots shown here are representative of three independent experiments with similar results, and, whereby the values above the figures represent relative density of the bands normalized to β-actin.

FIGS. 33-37 show the effect of fisetin treatment on induction of apoptosis in LNCaP cells as assessed by fluorecence microscopy, whereby the LNCaP cells were treated with fisetin (10-60 μM; 48 h), whereby the fluorescence was measured by a Zeiss 410 confocal microscope, and, whereby the data shown are from one representative experiment repeated two times with similar results, magnification ×400.

FIG. 38 is a bar graph showing the effect of fisetin treatment on induction of apoptosis in LNCaP cells, whereby apoptosis was determined by cell-death ELISA^{PLUS} as per the vendor's protocol.

FIG. 39-41 shows the effect of fisetin on cleavage of PARP, whereby the cells

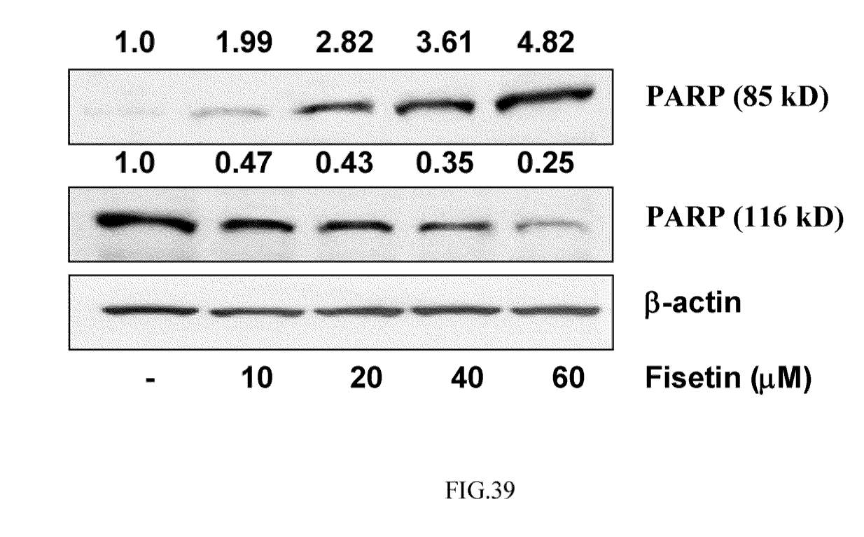

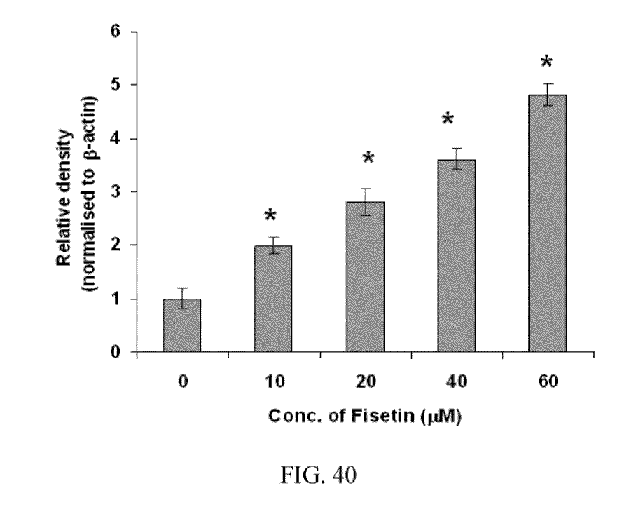

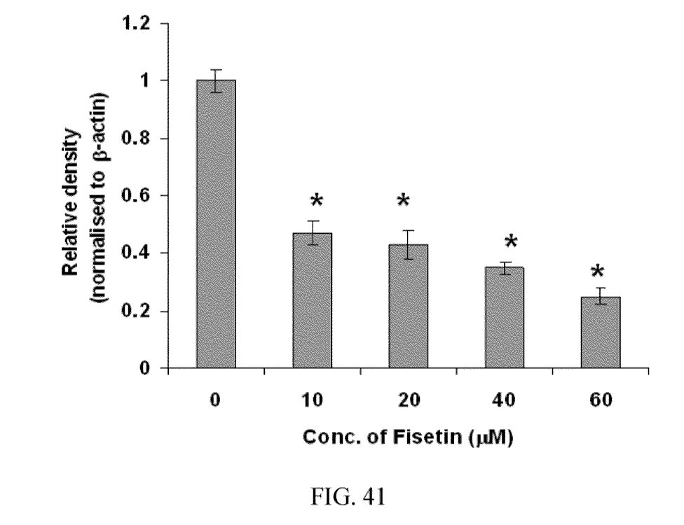

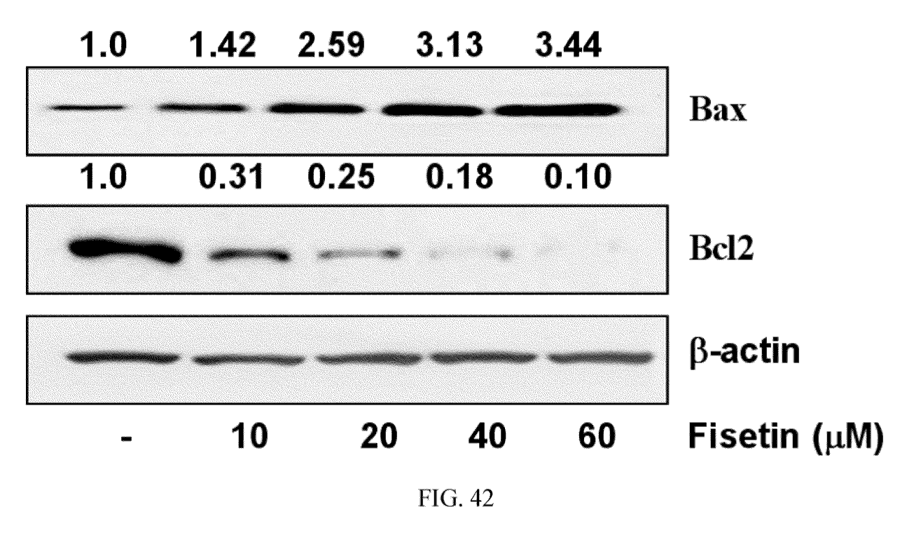

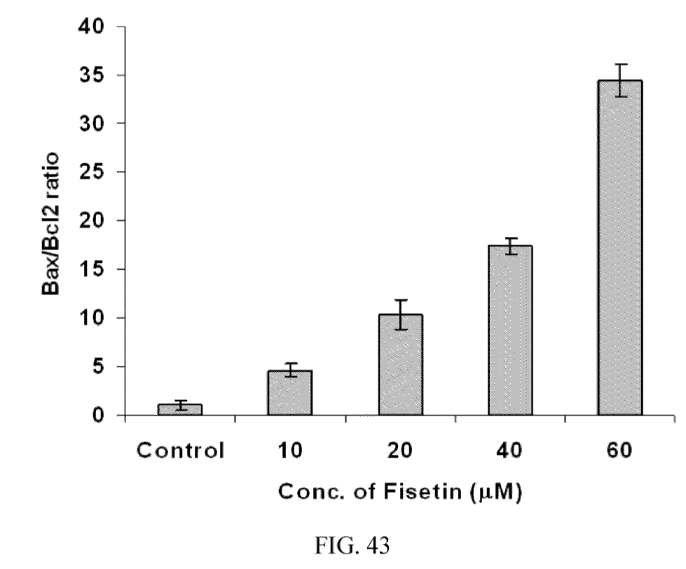

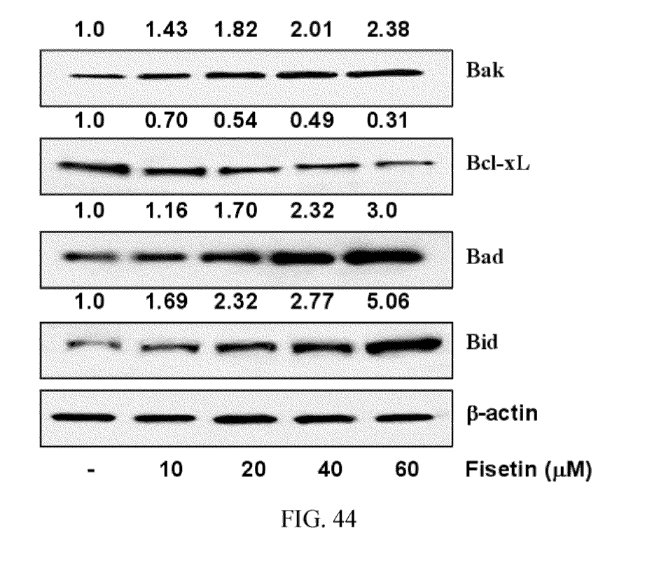

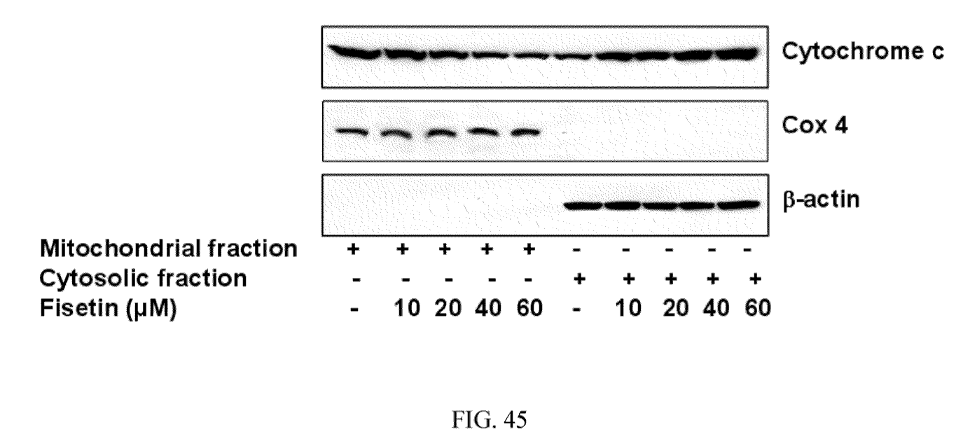

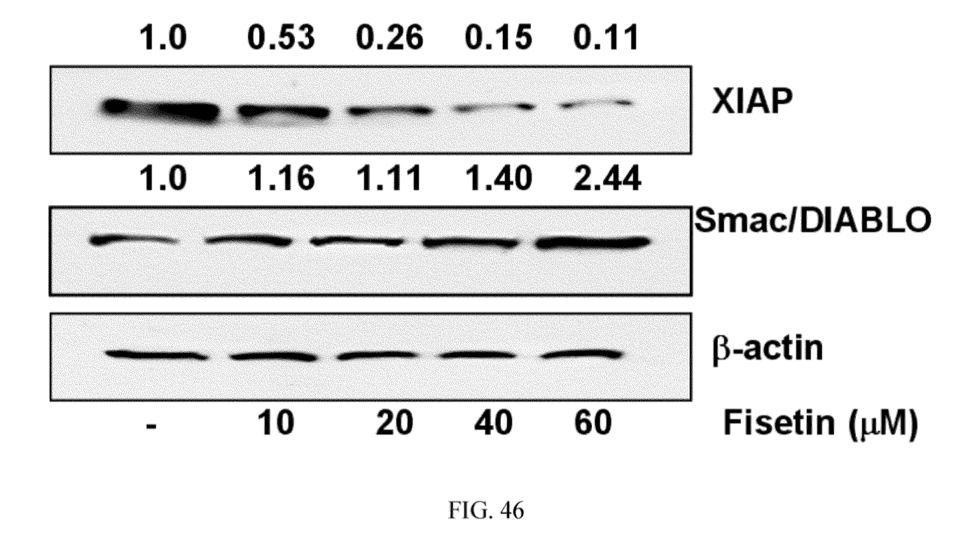

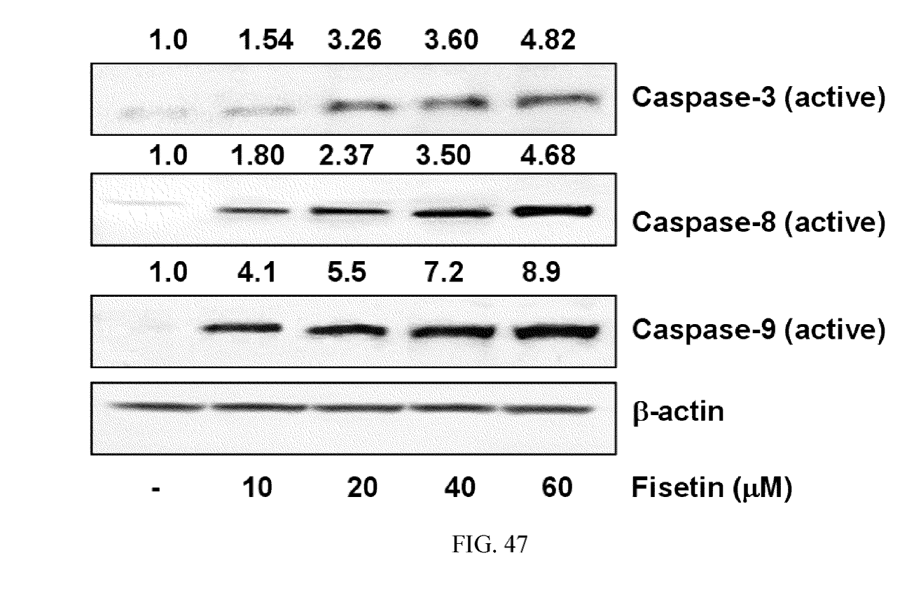

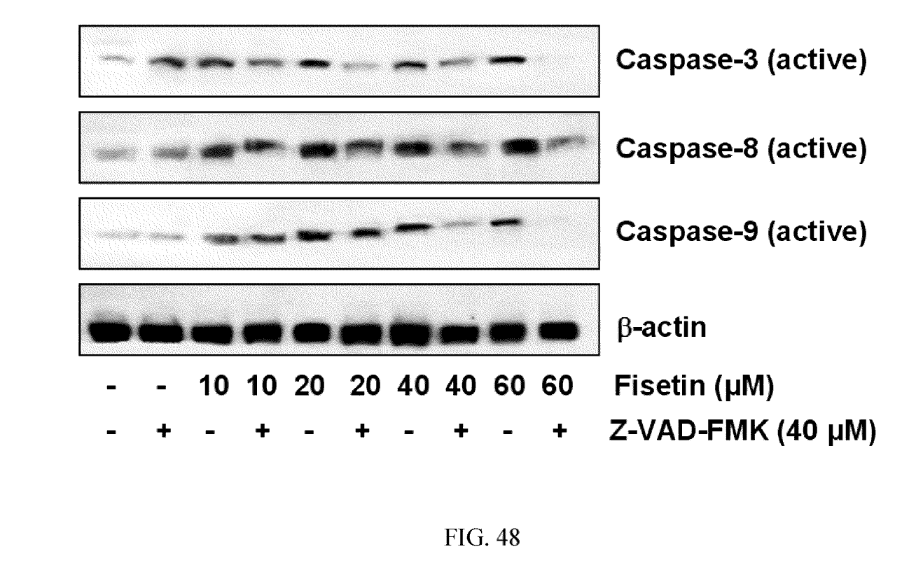

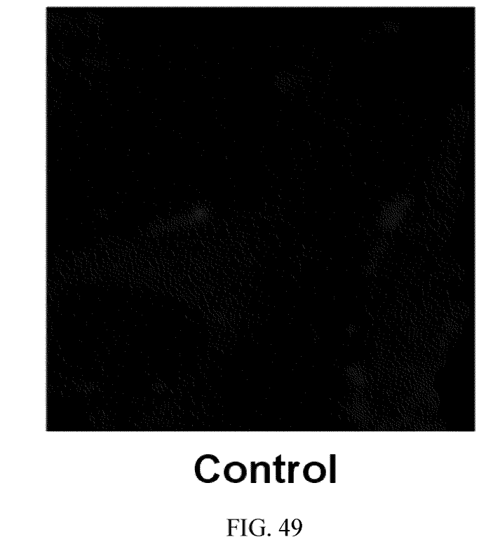

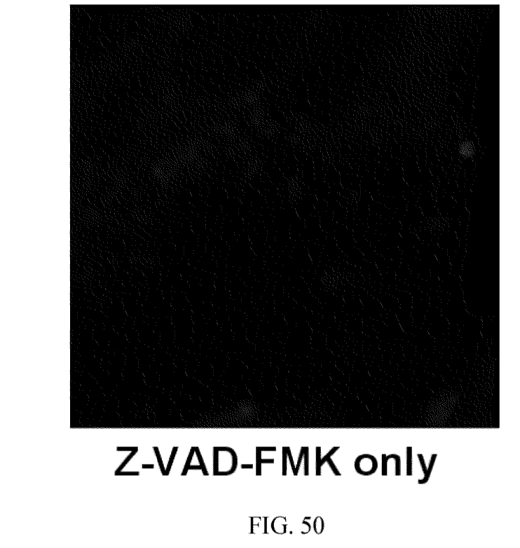

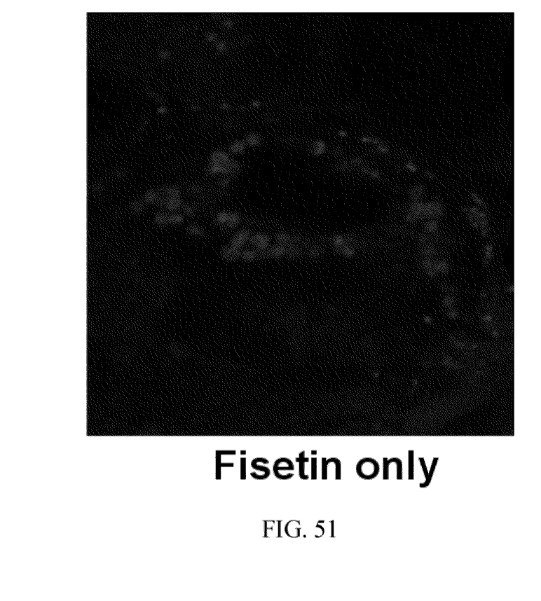

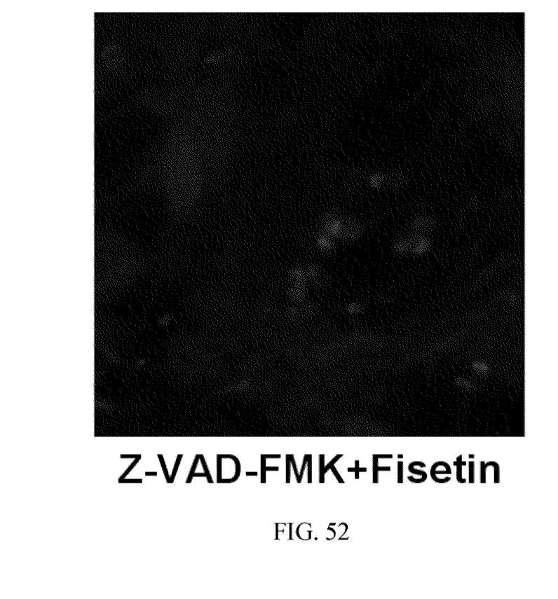

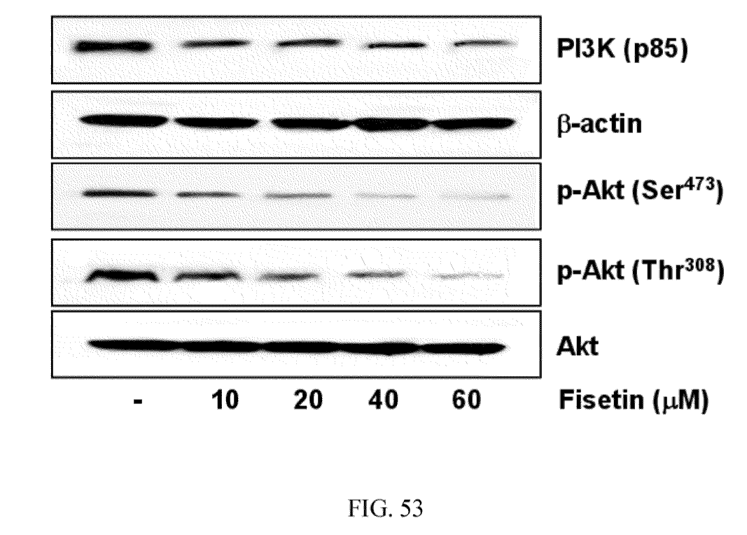

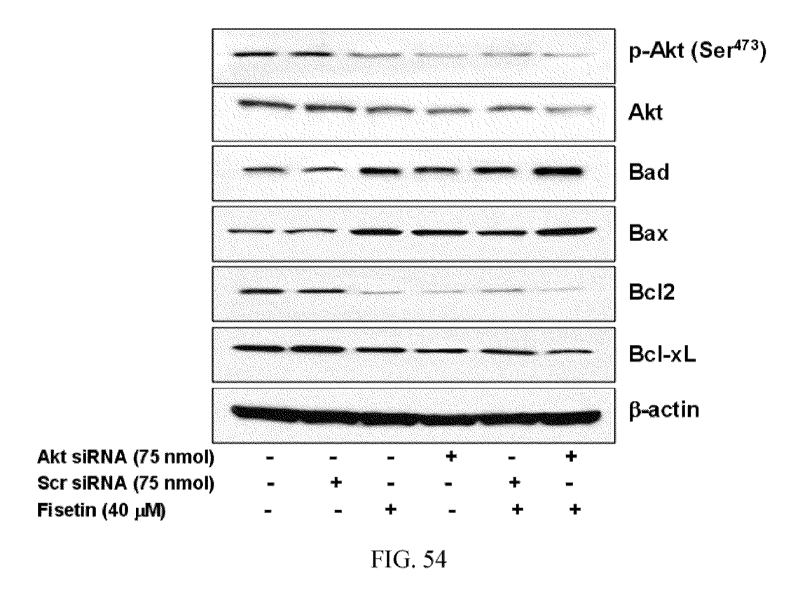

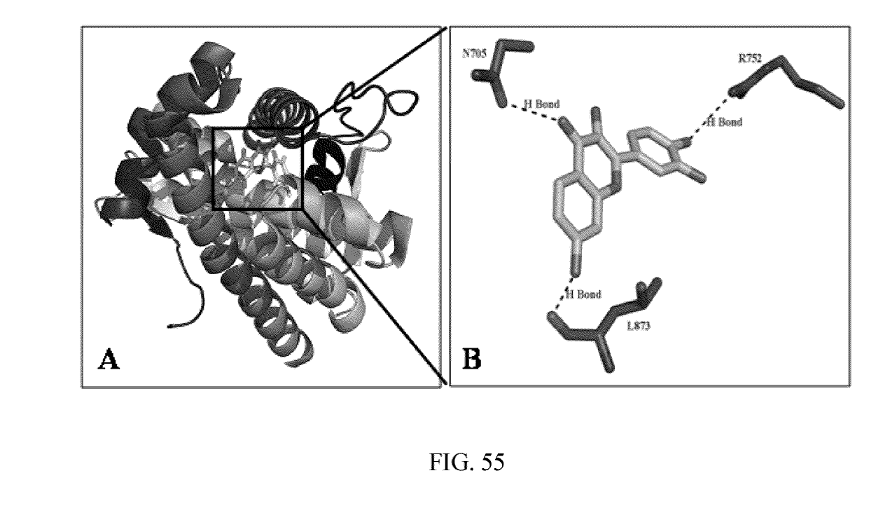

were treated with fisetin (10-60 μM; 48 h) and then harvested, whereby total cell lysates were prepared and 40 μg protein was subjected to SDS-PAGE followed by immunoblot analysis and chemiluminescence detection, whereby equal loading of protein was confirmed by stripping the immunoblot and reprobing it for β-actin, whereby the immunoblots shown are representative of three independent experiments with similar results, and, whereby the values above the figures represent relative density of the bands normalized to β-actin. FIGS. 40 and 41 are bar charts that document the relative density of bands shown in the immunoblots of FIG. 39. Results were analyzed using a two-tailed Student's t-test to assess statistical significance and P values <0.05 were considered significant.

FIG. 42 shows the effect of fisetin treatment of LNCaP cells on the protein expression of Bax and Bcl2, which are important key molecules involved in apoptosis and cell proliferation.

FIG. 43 is a bar graph showing the effect of fisetin treatment of LNCaP cells in terms of the Bax/Bcl2 ratio.

FIG. 44 shows the effect of fisetin treatment of LNCaP cells on protein expression of Bak, Bcl-xL, Bad and Bid, whereby the cells were treated with fisetin (10-60 μM; 48 h) and then harvested, whereby total cell lysates were prepared and 40 μg protein was subjected to SDS-PAGE followed by immunoblot analysis and chemiluminescence detection, whereby equal loading of protein was confirmed by stripping the immunoblot and reprobing it for β-actin, whereby the immunoblots shown are representative of three independent experiments with similar results, and, whereby the values above the figures represent relative density of the bands normalized to β-actin.

FIG. 45 shows the effect of fisetin treatment on mitochondrial release of cytochrome c into cytosol, whereby the cells were treated with fisetin (10-60 μM; 48 h) and then harvested, whereby mitochondrial and cytosolic fractions were prepared according to vendor's protocol and analyzed for cytochrome c and Cox-4 by immunoblot analysis and chemiluminescence detection, and, whereby the immunoblots shown are representative of 3 independent experiments with similar results.

FIG. 46 shows the effect of fisetin treatment on protein expression of XIAP and Smac/DIABLO in LNCaP cells, whereby the cells were treated with fisetin (10-60 μM; 48 h) and then harvested, whereby total cell lysates were prepared and 40 μg protein was subjected to SDS-PAGE followed by immunoblot analysis and chemiluminescence detection, whereby equal loading of protein was confirmed by stripping the immunoblot and reprobing it for β-actin, whereby the immunoblots shown are representative of three independent experiments with similar results, and, whereby the values above the figures represent relative density of the bands normalized to β-actin.

FIG. 47 shows the effect of fisetin treatment on protein expression of caspase-3, -8 and -9 in LNCaP cells, whereby the cells were treated with fisetin (10-60 μM; 48 h) and then harvested, whereby total cell lysates were prepared and 40 μg protein was subjected to SDS-PAGE followed by immunoblot analysis and chemiluminescence detection, whereby equal loading of protein was confirmed by stripping the immunoblot and reprobing it for β-actin, whereby the immunoblots shown are representative of three independent experiments with similar results, and, whereby the values above the figures represent relative density of the bands normalized to β-actin.

FIG. 48 shows the effect of Z-VAD-FMK on fisetin-induced activation of caspases in LNCaP cells, whereby the cells were incubated with 40 μM concentration of the general caspase inhibitor Z-VAD-FMK for 2 h followed by addition of fisetin (10-60 μM; 48 h) and then harvested.

FIGS. 49-52 show immunofluorescence staining for caspase-3 & 7, whereby LNCaP cells were incubated with 40 μM concentration of the general caspase inhibitor Z-VAD-FMK for 2 h followed by addition of fisetin (40 μM; 48 h) and then harvested, whereby caspase activity was detected within whole living cells using ICT's Magic Red™ substrate-based MR-Caspase assay kit and fluorescence photographs were obtained at 510-560 nm excitation and 610 nm emission, and, whereby the photomicrographs shown are from one representative experiment repeated two times with similar results.

FIG. 53 shows the inhibitory effects of fisetin on PI3K (p85) expression and phosphorylation of Akt (Ser473 and Thr308) in LNCaP cells, whereby the cells were treated with fisetin (10-60 μM; 48 h) and then harvested.

FIG. 54 shows Akt-dependent modulation in the Bcl2-family proteins by fisetin treatment in LNCaP cells, whereby the LNCaP cells were transfected with Akt-siRNA (75 nM) or scrambled siRNA (75 nM) and were then treated with 40 μM fisetin for 48 h, whereby whole cell lysate was prepared and 40 μg protein was subjected to SDS-PAGE followed by immunoblot analysis and chemiluminescence detection, whereby equal loading of protein was confirmed by stripping the immunoblot and reprobing it for β-actin, and, whereby the immunoblots shown are representative of 3 independent experiments with similar results.

FIG. 55 (Panels A and B) shows fisetin docked with the AR with a binding free energy of E=−7.46 Kcal/mole ˜3.4 μM, whereby fisetin formed hydrogen bonds with the Arg752 sidechain nitrogen, the Asn705 sidechain nitrogen, and, the Leu873 backbone oxygen, whereby such data was generated using AutoDock4.

### DETAILED DESCRIPTION OF THE EXEMPLARY EMBODIMENTS

Fisetin treatment, through modulations in cki-cyclin-cdk network, inhibits PI3K and Akt resulting in inhibition of cell growth followed by apoptosis of human prostate cancer LNCaP cells. The instant invention demonstrates the chemopreventive and chemotherapeutic potential of fisetin against PCa.

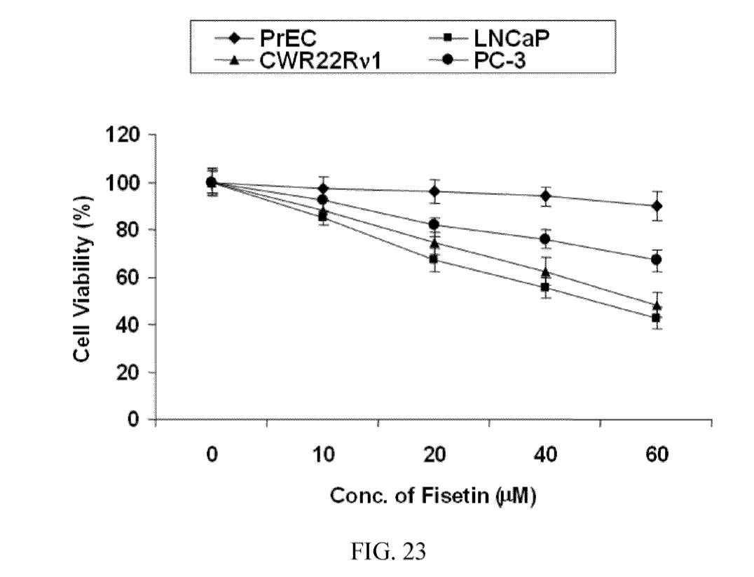

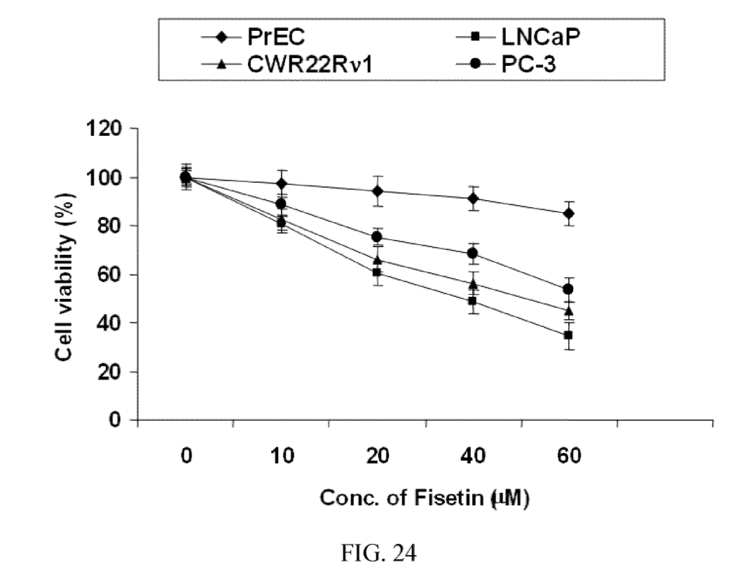

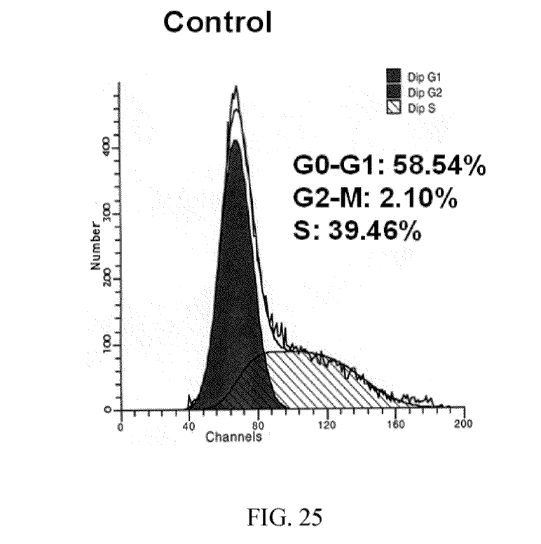

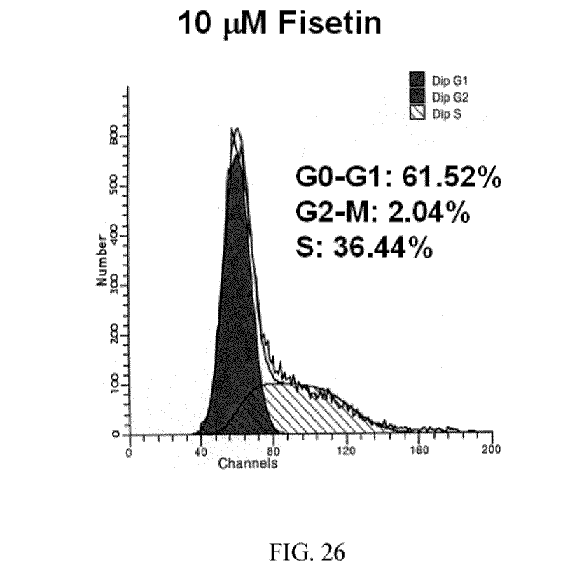

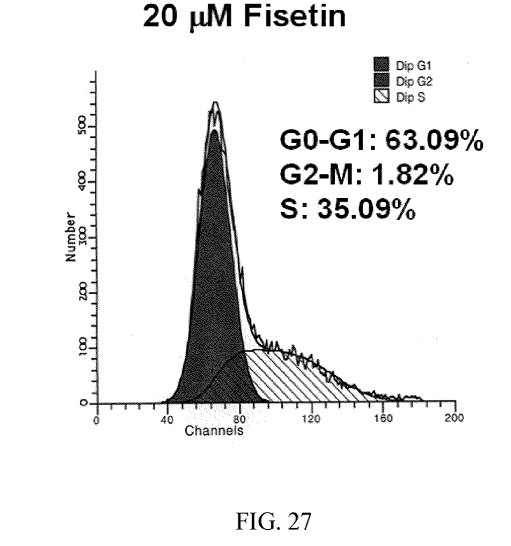

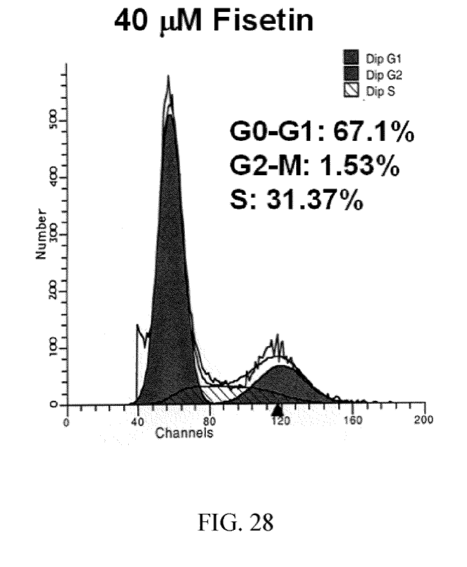

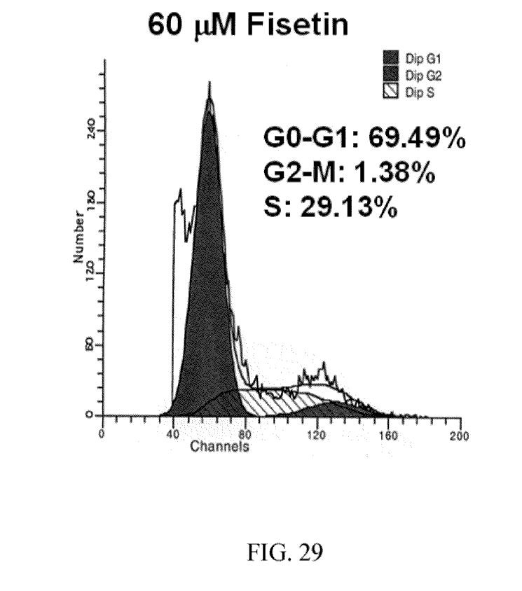

As shown in FIGS. 23 and 24, treatment of LNCaP, CWR22Ru1 and PC-3 cells with fisetin resulted in decrease in cell viability and had only minimal effect on PrEC cells. As shown in FIGS. 25-29, fisetin treatment of LNCaP cells also resulted in dose-dependent arrest of cells in G1 phase of the cell cycle. The involvement of cell cycle regulation-mediated apoptosis as a mechanism of cell growth inhibition has been reported. (Malik A et al., 2005, Pomegranate fruit juice for chemoprevention and chemotherapy of prostate cancer, Proc. Natl. Acad. Sci. U.S.A. 102:14813-14818; Kweon M H et al., 2007, A novel antioxidant 3-O-Caffeoyl-1-methylquinic acid enhances ultraviolet A-mediated apoptosis in immortalized HaCaT keratinocytes via Sp1-dependent transcriptional activation of p21(WAF1/Cip1) Oncogene 26:3559-3571; Sarfaraz S et al., 2006, Cannabinoid receptor agonist-induced apoptosis of human prostate cancer cells LNCaP proceeds through sustained activation of ERK1/2 leading to G1 cell cycle arrest, J. Biol. Chem. 281:39480-39491; Sato M et al., 2007, A natural peptide, dolastatin 15, induces G2/M cell cycle arrest and apoptosis of human multiple myeloma cells, Int. J Oncol 30:1453-9; and, Tian Z et al., 2008, Dulxanthone A induces cell cycle arrest and apoptosis via up-regulation of p53 through mitochondrial pathway in HepG2 cells, Int J Cancer 122:31-8).

The involvement of the cki-cyclin-cdk machinery during the induction of cell cycle arrest and apoptosis by fisetin in LNCaP cells was evaluated In eukaryotes, passage through the cell cycle is governed by the function of a family of protein kinase complexes. (Ahmad N et al., 1998, Photodynamic therapy results in induction of WAF1/CIP1/P21 leading to cell cycle arrest and apoptosis, Proc. Natl. Acad. Sci. USA 95:6977-6982). Each complex is minimally composed of a catalytic subunit, the cdk, and its essential activating partner, the cyclin.

Under normal conditions, these complexes are activated at specific intervals (and through a series of events) resulting in the progression of cells through different phases of cell cycle, thereby ensuring normal cell growth. (Sanchez I et al., 2005, New insights into cyclins, CDKs and cell cycle control, Semin. Cell Dev. Biol. 16:311-321). Any defect in this machinery causes an altered cell cycle regulation that may result in unwanted cellular proliferation ultimately culminating in the development of cancer.

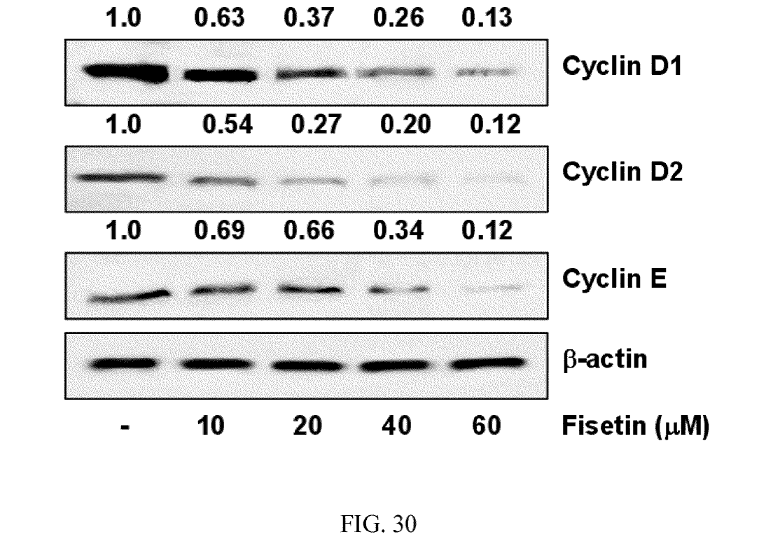

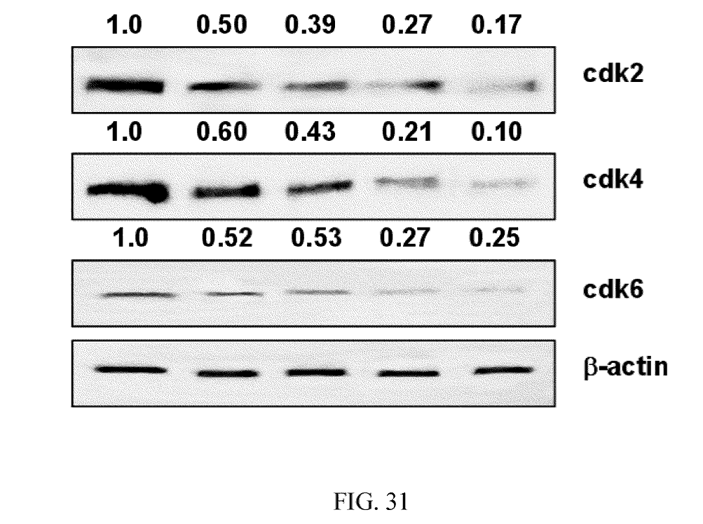

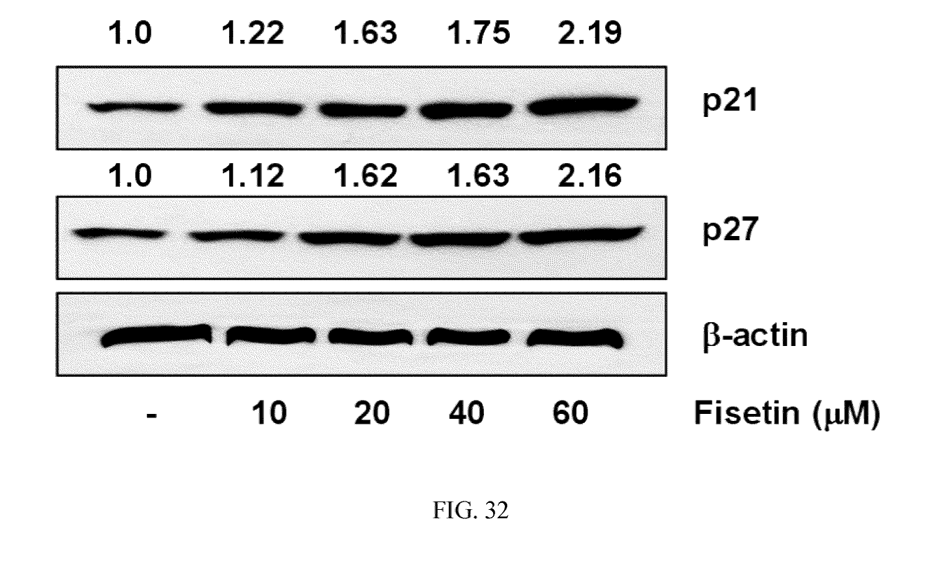

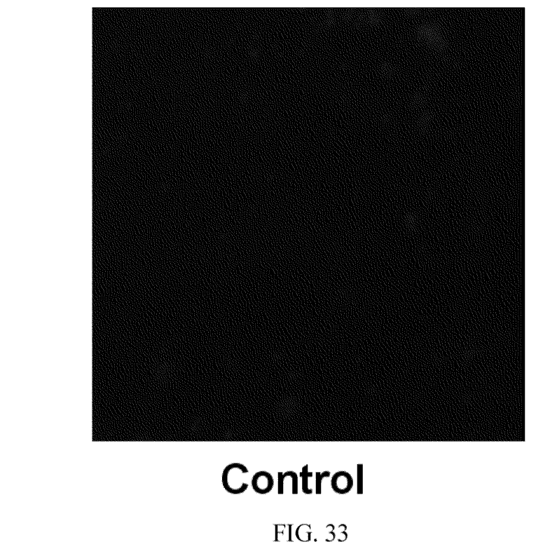

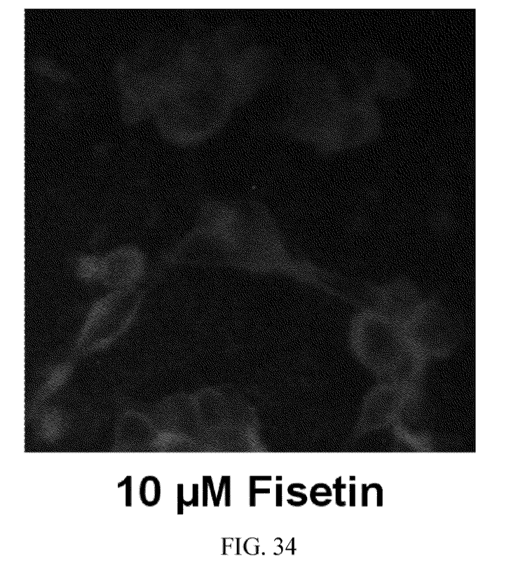

The instant invention demonstrates that fisetin treatment of LNCaP cells resulted in a G1 phase arrest of the cell cycle. The effect of fisetin on cell cycle-regulatory molecules operative in the G1 phase of the cell cycle was also evaluated. As shown in FIGS. 30 and 31, fisetin treatment of the cells resulted in significant down-modulation of cyclins and cdks. As shown in FIG. 32, the data also demonstrates a significant up-regulation of the cki WAF1/p21 and KIP1/p27 by fisetin.

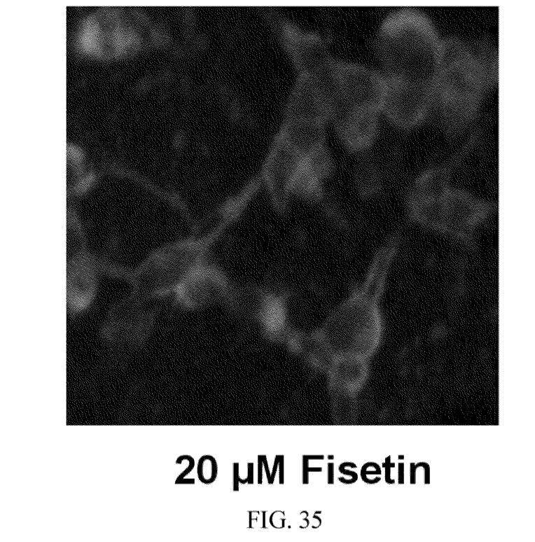

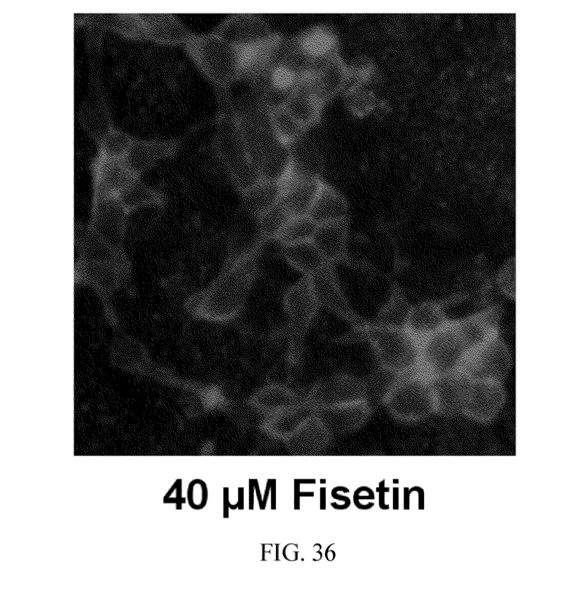

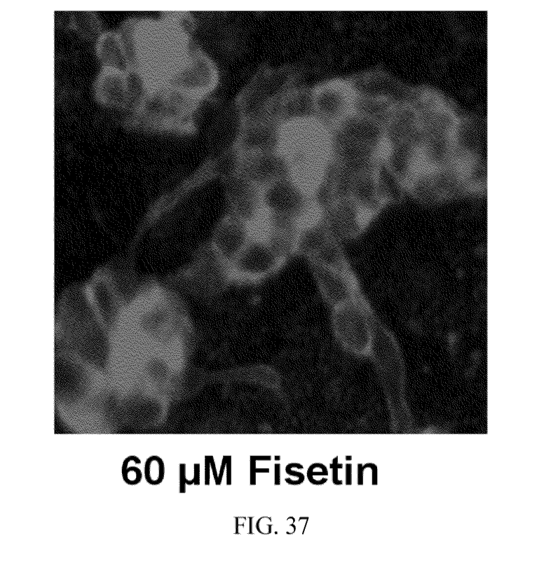

The induction of apoptosis is a physiological process that functions as an essential mechanism of tissue homeostasis and is regarded as the preferred way to eliminate unwanted cells. As shown in FIGS. 33-41, this observation was verified by fluorescence microscopy and PARP cleavage. Collectively, these results suggest that fisetin inhibits the growth of prostate carcinoma cells through cell cycle arrest and induction of apoptosis.

Members of the Bcl-2 family of proteins are critical regulators of the apoplotic pathway. Bcl-2 is an upstream effector molecule in the apoptotic pathway and is identified as a potent suppressor of apoptosis. (Oltersdorf T et al., 2005, An inhibitor of Bcl-2 family proteins induces regression of solid tumours, Nature 435:677-681). Bcl-2 has been shown to form a heterodimer with the pro-apoptotic member Bax, therefore, it may neutralize its pro-apoptotic effects.

Alterations in the levels of Bax and Bcl-2 (i.e. the ratio of Bax/Bcl-2) is a critical factor and plays an important role in determining whether cells will undergo apoptosis under experimental conditions that promote cell death. As shown in FIG. 42, a decrease in Bcl-2 and increase in Bax protein expression was observed in LNCaP cells, and, the ratio of Bax to Bcl-2 was altered in favor of apoptosis. (See FIG. 43). Up-regulation of Bax and down-modulation of Bcl-2 may be another molecular mechanism through which fisetin induces apoptosis. As shown in FIG. 44, upregulation in the protein expressions of proapoptotic Bak. Bad and Bid and down regulation of antiapoptotic Bcl-xL is also observed upon treatment with fisetin in LNCaP cells.

Mitochondria may contain and release proteins such as cytochrome c that are involved in the apoptotic cascade. Cell-free systems demonstrate that mitochondrial products are rate limiting for the activation of caspases and endonucleases in cell extracts. (Martinou J C et al., 2000, Cytochrome c release from mitochondria: all or nothing, Nat. Cell Biol. 2: E41-E43). Functional studies indicate that drug-induced opening or closing of the mitochondrial megachannel (permeability transition pore) can induce or prevent apoptosis. (Tsujimoto Y et al., 2000, Bcl-2 family: life-or-death switch. FEBS Lett. 466:6-10).

These experiments indicate that cytochrome c is a key factor in apoptosis, and that its release further activates caspases resulting in the appearance of apoptosis. As shown in FIG. 45, the instant invention confirms that cytosolic cytochrome c was increased in LNCaP cells after treatment with fisetin. Suppression of apoptosis promotes tumor progression, immune evasion of neoplastic cells as well as resistance to chemotherapy and irradiation. (Igney F H et al., 2002, Death and anti-death: tumour resistance to apoptosis, Nat. Rev. Cancer 2:277-288).

Several genes critical in the regulation of apoptosis have been identified, including X-linked inhibitor of apoptosis (XIAP)—a member of the inhibitor of apoptosis (IAP) family. XIAP is thought to act as a key determinant of apoptosis resistance by effectively inhibiting the activation of caspase-3, -7 and -9. Thus, high expression of XIAP has been reported in many malignant tumor types, such as carcinomas of the breast, ovaries, lung, pancreas, cervix and prostate. (Hofmann H S et al., 2002, Expression of inhibitors of apoptosis (IAP) proteins in non-small cell human lung cancer, J. Cancer Res. Clin. Oncol. 128:554-560).

Whereas antiapoptotic XIAP has been shown to be a potent caspase inhibitor (Suzuki Y et al., 2001, Ubiquitin-protein ligase activity of X-linked inhibitor of apoptosis protein promotes proteasomal degradation of caspase-3 and enhances its anti-apoptotic effect in Fas-induced cell death, Proc. Natl. Acad. Sci. USA 98:8662-866730), its antagonists Smac/DIABLO and Omi/HtrA2 promote apoptosis by binding to XIAP thereby preventing them from inhibition of caspases. Recently, an inverse relation between XIAP expression and Smac/DIABLO has been observed after apoptosis induction in colon cancer cells, lymphoma cells and keratinocytes. (Takasawa R et al., 2003, Sustained release of Smac/DIABLO from mitochondria commits to undergo UVB-induced apoptosis. Apoptosis 8:291-299). As shown in FIG. 46, fisetin treatment (10-60 μM: 48 h) of LNCaP cells caused inhibition of XIAP and induction of Smac/DIABLO.

Caspases are comprised of 12 proteins and are a family of cysteinyl aspartate-specific proteases involved in apoptosis and are subdivided into initiator (caspases 8, 9, 10) and executioner (caspases 3, 6, 7) caspases. The intrinsic and extrinsic pathways converge at caspase-3. Active caspase 9 and caspase 8 of the intrinsic and extrinsic pathways, respectively, have been shown to directly cleave and activate the effector protease caspase-3.

As shown in FIG. 47, caspase-3, -8 and -9are activated during fisetin-mediated apoptosis. As shown in FIGS. 48-52, immunoblot analysis and immunofluorescence staining demonstrate that addition of Z-VAD-FMK (a general caspase inhibitor) significantly reduced fisetin-mediated apoptosis further demonstrating that fisetin-mediated apoptosis in LNCaP cells was caspase-dependent.

The phosphatidyl inositol 3-kinase (PI3K)/Akt pathway is activated downstream of a variety of extracellular signals and activation of this signaling pathway impacts a number of cellular processes including cell growth, proliferation and survival. The alteration of components of this pathway, through either activation of oncogenes or inactivation of tumor suppressors, disrupts a signaling equilibrium and can thus lead to cellular transformation. (Hanahan D et al., 2000. The hallmarks of cancer, Cell 100:57-70).

As shown in FIG. 53, treatment of LNCaP cells with fisetin caused a decrease in the protein expression of PI3K (p85) and phosphorylation of Akt at both Thr308 and Ser473. The Akt family has been shown to be the primary downstream mediator of the effects of PI3K and regulates a variety of cellular processes through the phosphorylation of a wide spectrum of downstream substrates. Dysregulation of the PI3K/Akt signaling pathway can lead to an alteration of all the aspects of cell physiology that comprise the hallmarks of cancer.

Cell survival is influenced by Akt through a variety of effector proteins

including inhibition of the proapoptotic Bcl2 family member Bad and inhibition of the forkhead transcription factors which normally activate apoptotsis related genes. As shown in FIG. 54, silencing of Akt by siRNA caused an increase in the protein expressions of Bad and Bax and decrease in Bcl2 and Bcl-xL, which was further augmented upon treatment with fisetin suggesting that these effects are mediated in part through Akt.

The instant invention demonstrates that treatment of LNCaP cells with Fisetin (a dietary flavonoid) caused inhibition of PCa by G1 phase cell cycle arrest, modulating cki-cyclin-cdk network and induction of apoptosis, therefore, fisetin is a useful chemotherapeutic agent against prostate cancer.

As used herein, “prodrugs” are compounds that are pharmacologically inert but are converted by enzyme or chemical action to an active form of the drug (i.e., an active pharmaceutical ingredient) at or near the predetermined target site. In other words, prodrugs are inactive compounds that yield an active compound upon metabolism in the body, which may or may not be enzyme controlled. Prodrugs may also be broadly classified into two groups: bioprecursor and carrier prodrugs. Prodrugs may also be subclassified according to the nature of their action. Bioprecursor prodrugs are compounds that already contain the embryo of the active species within their structure, whereby the active species are produced upon metabolism. For example, the first prodrug, antibacterial prontosil, is metabolized in vivo to its active metabolite sulphanilamide. Carrier prodrugs are formed by combining the active drug with a carrier species forming a compound having desirable chemical and biological characteristics, whereby the link is an ester or amide so that the carrier prodrug is easily metabolized upon absorption or delivery to the target site. For example, lipophilic moieties may be incorporated to improve transport through membranes. Carrier prodrugs linked by a functional group to carrier are referred to as bipartate prodrugs. Prodrugs where the carrier is linked to the drug by a separate structure are referred to as tripartate prodrugs, whereby the carrier is removed by an enzyme-controlled metabolic process, and whereby the linking structure is removed by an enzyme system or by a chemical reaction. (Thomas G, Medicinal Chemistry: An Introduction, 2000, John Wiley & Sons. Ltd. pp. 12, 17, 243 and 364-372)(See also, Wermuth C G, 2003, The Practice of Medicinal Chemistry, 2nd Ed., Academic Press 33:561-582). The phrase “hydroxy-protecting group” refers to any suitable group, such as tert-butyloxy-carbonyl (t-BOC) and t-butyl-dimethyl-silyl (TBS). Other hydroxy protecting groups are shown in Hanson J R, 1999, Protecting Groups in Organic Synthesis, Sheffield Academic Press, 2:8-35, which is incorporated herein by reference.

As used herein, “salts” of the instant compound may be a pharmaceutically suitable (i.e., pharmaceutically acceptable) salt including, but not limited to, acid addition salts formed by mixing a solution of the instant compound with a solution of a pharmaceutically acceptable acid. The pharmaceutically acceptable acid may be hydrochloric acid, methanesulphonic acid, fumaric acid, maleic acid, succinic acid, acetic acid, benzoic acid, oxalic acid, citric acid, tartaric acid, carbonic acid or phosphoric acid. Various pharmaceutically acceptable salts are well known in the art and may be used with the instant compound such as those disclosed in Berge S M et al., Pharmaceutical Salts. J. Pharm. Sci. 66:1-19 (1977) and Haynes D A et al., Occurrence of pharmaceutically acceptable anions and cations in the Cambridge Structural Database. J. Pharm. Sci. 94:2111-2120 (2005), which are hereby incorporated herein by reference. For example, the list of FDA-approved commercially marketed salts includes acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, mitrate, pamoate, pantothenate, phosphate, diphosphate, polygalacturonate, salicylate, stearate, subacetate, succinate, sulfate, tannate, tartrate, teoclate, and triethiodide.

As used herein, “hydrates” of the instant compound may be a pharmaceutically suitable (i.e., pharmaceutically acceptable) hydrate that is a compound formed by the addition of water or its elements to a host molecule (e.g., the free form version of the compound) including, but not limited to, monohydrates, dihydrates, etc.

As used herein, “solvates” of the instant compound may be a pharmaceutically suitable (i.e., pharmaceutically acceptable) solvate, whereby solvation is an interaction of a solute with the solvent which leads to stabilization of the solute species in the solution, and whereby the solvated state is an ion in a solution complexed by solvent molecules. Solvates and hydrates may also be referred to as “analogues.”

The active pharmaceutical ingredient, Fisetin (3,3′,4′,7-tetrahydroxyflavone, also referred to as fisetic acid), may be administered via various pharmaceutically suitable dosage forms.

Pharmaceutically suitable topical and oral carrier systems (also referred to as drug delivery systems, which are modern technology, distributed with or as a part of a drug product that allows for the uniform release or targeting of drugs to the body) preferably include FDA-approved and/or USP-approved inactive ingredients. Under 21 CFR 210.3(b)(8), an inactive ingredient is any component of a drug product other than the active ingredient. According to 21 CFR 210.3(b)(7), an active ingredient is any component of a drug product intended to furnish pharmacological activity or other direct effect in the diagnosis, cure, mitigation, treatment, or prevention of disease, or to affect the structure or any function of the body of humans or other animals. Active ingredients include those components of the product that may undergo chemical change during the manufacture of the drug product and be present in the drug product in a modified form intended to furnish the specified activity or effect. As used herein, a kit (also referred to as a dosage form) is a packaged collection of related material.

As used herein, the topical dosage form includes various dosage forms known in the art such as lotions (an emulsion, liquid dosage form, whereby this dosage form is generally for external application to the skin), lotion augmented (a lotion dosage form that enhances drug delivery, whereby augmentation does not refer to the strength of the drug in the dosage form), gels (a semisolid dosage form that contains a gelling agent to provide stiffness to a solution or a colloidal dispersion, whereby the gel may contain suspended particles), ointments (a semisolid dosage form, usually containing <20% water and volatiles and >50% hydrocarbons, waxes, or polyols as the vehicle, whereby this dosage form is generally for external application to the skin or mucous membranes), ointment augmented (an ointment dosage form that enhances drug delivery, whereby augmentation does not refer to the strength of the drug in the dosage form), creams (an emulsion, semisolid dosage form, usually containing >20% water and volatiles and/or <50% hydrocarbons, waxes, or polyols as the vehicle, whereby this dosage form is generally for external application to the skin or mucous membranes), cream augmented (a cream dosage form that enhances drug delivery, whereby augmentation does not refer to the strength of the drug in the dosage form), emulsion (a dosage form consisting of a two-phase system comprised of at least two immiscible liquids, one of which is dispersed as droplets, internal or dispersed phase, within the other liquid, external or continuous phase, generally stabilized with one or more emulsifying agents, whereby emulsion is used as a dosage form term unless a more specific term is applicable, e.g. cream, lotion, ointment), suspensions (a liquid dosage form that contains solid particles dispersed in a liquid vehicle), suspension extended release (a liquid preparation consisting of solid particles dispersed throughout a liquid phase in which the particles are not soluble; the suspension has been formulated in a manner to allow al least a reduction in dosing frequency as compared to that drug presented as a conventional dosage form, e.g., as a solution or a prompt drug-releasing, conventional solid dosage form), pastes (a semisolid dosage form, containing a large proportion, 20-50%, of solids finely dispersed in a fatty vehicle, whereby this dosage form is generally for external application to the skin or mucous membranes), solutions (a clear, homogeneous liquid dosage form that contains one or more chemical substances dissolved in a solvent or mixture of mutually miscible solvents), powders (an intimate mixture of dry, finely divided drugs and/or chemicals that may be intended for internal or external use), shampoos (a lotion dosage form which has a soap or detergent that is usually used to clean the hair and scalp; it is often used as a vehicle for dermatologic agents), shampoo suspensions (a liquid soap or detergent containing one or more solid, insoluble substances dispersed in a liquid vehicle that is used to clean the hair and scalp and is often used as a vehicle for dermatologic agents), aerosol foams (a dosage form containing one or more active ingredients, surfactants, aqueous or nonaqueous liquids, and the propellants; if the propellant is in the internal discontinuous phase, i.e., of the oil-in-water type, a stable foam is discharged, and if the propellant is in the external continuous phase, i.e., of the water-in-oil type, a spray or a quick-breaking foam is discharged), sprays (a liquid minutely divided as by a jet of air or steam), metered spray (a non-pressurized dosage form consisting of valves which allow the dispensing of a specified quantity of spray upon each activation), suspension spray (a liquid preparation containing solid particles dispersed in a liquid vehicle and in the form of coarse droplets or as finely divided solids to be applied locally, most usually to the nasal-pharyngeal tract, or topically to the skin), jellies (a class of gels, which are semisolid systems that consist of suspensions made up of either small inorganic particles or large organic molecules interpenetrated by a liquid, in which the structural coherent matrix contains a high portion of liquid, usually water), films (a thin layer or coating), film extended release (a drug delivery system in the form of a film that releases the drug over an extended period in such a way as to maintain constant drug levels in the blood or target tissue), film soluble (a thin layer or coating which is susceptible to being dissolved when in contact with a liquid), sponges (a porous, interlacing, absorbent material that contains a drug, whereby it is typically used for applying or introducing medication, or for cleansing, and whereby a sponge usually retains its shape), swabs (a small piece of relatively flat absorbent material that contains a drug, whereby a swab may also be attached to one end of a small stick, and whereby a swab is typically used for applying medication or for cleansing), patches (a drug delivery system that often contains an adhesive backing that is usually applied to an external site on the body, whereby its ingredients either passively diffuse from, or are actively transported from, some portion of the patch, whereby depending upon the patch, the ingredients are either delivered to the outer surface of the body or into the body, and whereby a patch is sometimes synonymous with the terms ‘extended release film’ and ‘system’), patch extended release (a drug delivery system in the form of a patch that releases the drug in such a manner that a reduction in dosing frequency compared to that drug presented as a conventional dosage form, e.g., a solution or a prompt drug-releasing, conventional solid dosage form), patch extended release electronically controlled (a drug delivery system in the form of a patch which is controlled by an electric current that releases the drug in such a manner that a reduction in dosing frequency compared to that drug presented as a conventional dosage form, e.g., a solution or a prompt drug-releasing, conventional solid dosage form), and the like. The various topical dosage forms may also be formulated as immediate release, controlled release, sustained release, or the like.

The topical dosage form composition contains an active pharmaceutical

ingredient and one or more inactive pharmaceutical ingredients such as excipients, colorants, pigments, additives, fillers, emollients, surfactants (e.g., anionic, cationic, amphoteric and nonionic), penetration enhancers (e.g., alcohols, fatty alcohols, fatty acids, fatty acid esters and polyols), and the like. Various FDA-approved topical inactive ingredients are found at the FDA's “The Inactive Ingredients Database” that contains inactive ingredients specifically intended as such by the manufacturer, whereby inactive ingredients can also be considered active ingredients under certain circumstances, according to the definition of an active ingredient given in 21 CFR 210.3(b)(7). Alcohol is a good example of an ingredient that may be considered either active or inactive depending on the product formulation.

As used herein, the oral dosage form includes capsules (a solid oral dosage

form consisting of a shell and a filling, whereby the shell is composed of a single sealed enclosure, or two halves that fit together and which are sometimes sealed with a band, and whereby capsule shells may be made from gelatin, starch, or cellulose, or other suitable materials, may be soft or hard, and are filled with solid or liquid ingredients that can be poured or squeezed), capsule or coated pellets (solid dosage form in which the drug is enclosed within either a hard or soft soluble container or “shell” made from a suitable form of gelatin; the drug itself is in the form of granules to which varying amounts of coating have been applied), capsule coated extended release (a solid dosage form in which the drug is enclosed within either a hard or soft soluble container or “shell” made from a suitable form of gelatin; additionally, the capsule is covered in a designated coating, and which releases a drug or drugs in such a manner to allow at least a reduction in dosing frequency as compared to that drug or drugs presented as a conventional dosage form), capsule delayed release (a solid dosage form in which the drug is enclosed within either a hard or soft soluble container made from a suitable form of gelatin, and which releases a drug (or drugs) at a time other than promptly after administration, whereby enteric-coated articles are delayed release dosage forms), capsule delayed release pellets (solid dosage form in which the drug is enclosed within either a hard or soft soluble container or “shell” made from a suitable form of gelatin); the drug itself is in the form of granules to which enteric coating has been applied, thus delaying release of the drug until its passage into the intestines), capsule extended release (a solid dosage form in which the drug is enclosed within either a hard or soft soluble container made from a suitable form of gelatin, and which releases a drug or drugs in such a manner to allow a reduction in dosing frequency as compared to that drug or drugs presented as a conventional dosage form), capsule film-coated extended release (a solid dosage form in which the drug is enclosed within either a hard or soft soluble container or “shell” made from a suitable form of gelatin; additionally, the capsule is covered in a designated film coating, and which releases a drug or drugs in such a manner to allow at least a reduction in dosing frequency as compared to that drug or drugs presented as a conventional dosage form), capsule gelatin coated (a solid dosage form in which the drug is enclosed within either a hard or soft soluble container made from a suitable form of gelatin; through a banding process, the capsule is coated with additional layers of gelatin so as to form a complete seal), capsule liquid filled (a solid dosage form in which the drug is enclosed within a soluble, gelatin shell which is plasticized by the addition of a polyol, such as sorbitol or glycerin, and is therefore of a somewhat thicker consistency than that of a hard shell capsule; typically, the active ingredients are dissolved or suspended in a liquid vehicle), granule (a small particle or grain), pellet (a small sterile solid mass consisting of a highly purified drug, with or without excipients, made by the formation of granules, or by compression and molding), pellets coaled extended release (a solid dosage form in which the drug itself is in the form of granules to which varying amounts of coating have been applied, and which releases a drug or drugs in such a manner to allow a reduction in dosing frequency as compared to that drug or drugs presented as a conventional dosage form), pill (a small, round solid dosage form containing a medicinal agent intended for oral administration), powder (an intimate mixture of dry, finely divided drugs and/or chemicals that may be intended for internal or external use), elixir (a clear, pleasantly flavored, sweetened hydroalcoholic liquid containing dissolved medicinal agents; it is intended for oral use), chewing gum (a sweetened and flavored insoluble plastic material of various shapes which when chewed, releases a drug substance into the oral cavity), syrup (an oral solution containing high concentrations of sucrose or other sugars; the term has also been used to include any other liquid dosage form prepared in a sweet and viscid vehicle, including oral suspensions), tablet (a solid dosage form containing medicinal substances with or without suitable diluents), tablet chewable (a solid dosage form containing medicinal substances with or without suitable diluents that is intended to be chewed, producing a pleasant tasting residue in the oral cavity that is easily swallowed and does not leave a bitter or unpleasant after-taste), tablet coated (a solid dosage form that contains medicinal substances with or without suitable diluents and is covered with a designated coaling), tablet coated particles (a solid dosage form containing a conglomerate of medicinal particles that have each been covered with a coating), tablet delayed release (a solid dosage form which releases a drug or drugs at a time other than promptly after administration, whereby enteric-coated articles are delayed release dosage forms), tablet delayed release particles (a solid dosage form containing a conglomerate of medicinal particles that have been covered with a coating which releases a drug or drugs at a time other than promptly after administration, whereby enteric-coated articles are delayed release dosage forms), tablet dispersible (a tablet that, prior to administration, is intended to be placed in liquid, where its contents will be distributed evenly throughout that liquid, whereby term ‘tablet, dispersible’ is no longer used for approved drug products, and it has been replaced by the term ‘tablet, for suspension’), tablet effervescent (a solid dosage form containing mixtures of acids, e.g., citric acid, tartaric acid, and sodium bicarbonate, which release carbon dioxide when dissolved in water, whereby it is intended to be dissolved or dispersed in water before administration), tablet extended release (a solid dosage form containing a drug which allows at least a reduction in dosing frequency as compared to that drug presented in conventional dosage form), tablet film coated (a solid dosage form that contains medicinal substances with or without suitable diluents and is coaled with a thin layer of a water-insoluble or water-soluble polymer), tablet film coated extended release (a solid dosage form that contains medicinal substances with or without suitable diluents and is coated with a thin layer of a water-insoluble or water-soluble polymer; the tablet is formulated in such manner as to make the contained medicament available over an extended period of time following ingestion), tablet for solution (a tablet that forms a solution when placed in a liquid), tablet for suspension (a tablet that forms a suspension when placed in a liquid, which is formerly referred to as a ‘dispersible tablet’), tablet multilayer (a solid dosage form containing medicinal substances that have been compressed to form a multiple-layered tablet or a table-within-a-table, the inner tablet being the core and the outer portion being the shell), tablet multilayer extended release (a solid dosage form containing medicinal substances that have been compressed to form a multiple-layered tablet or a tablet-within-a-tablet, the inner tablet being the core and the outer portion being the shell, which, additionally, is covered in a designated coating; the tablet is formulated in such manner as to allow at least a reduction in dosing frequency as compared to that drug presented as a conventional dosage form), tablet orally disintegrating (a solid dosage form containing medicinal substances which disintegrates rapidly, usually within a matter of seconds, when placed upon the tongue), tablet orally disintegrating delayed release (a solid dosage form containing medicinal substances which disintegrates rapidly, usually within a matter of seconds, when placed upon the tongue, but which releases a drug or drugs at a time other than promptly after administration), tablet soluble (a solid dosage form that contains medicinal substances with or without suitable diluents and possesses the ability to dissolve in fluids), tablet sugar coated (a solid dosage form that contains medicinal substances with or without suitable diluents and is coated with a colored or an uncolored water-soluble sugar), osmotic, and the like.

The oral dosage form composition contains an active pharmaceutical ingredient and one or more inactive pharmaceutical ingredients such as diluents, solubilizers, alcohols, binders, controlled release polymers, enteric polymers, disintegrants, excipients, colorants, flavorants, sweeteners, antioxidants, preservatives, pigments, additives, fillers, suspension agents, surfactants (e.g., anionic, cationic, amphoteric and nonionic), and the like. Various FDA-approved topical inactive ingredients are found at the FDA's “The Inactive Ingredients Database” that contains inactive ingredients specifically intended as such by the manufacturer, whereby inactive ingredients can also be considered active ingredients under certain circumstances, according to the definition of an active ingredient given in 21 CFR 210.3(b)(7). Alcohol is a good example of an ingredient that may be considered either active or inactive depending on the product formulation.

### EXAMPLES

Materials. Anti-cyclins D1, D2, E, Bad, Bcl-xL, active caspases -3, -8 and -9,

Akt, phospho-Akt (Ser473 and Thr 308), PI3K (p85), XIAP, Smac/DIABLO antibodies were obtained from Cell Signaling Technology (Beverly, Mass.). The mono and polyclonal antibodies cdks 2, 4 and 6, WAF1/p21, KIP1/p27 and Bcl2 were obtained from Santa Cruz Biotechnology Inc. (Santa Cruz, Calif.). PARP (116) and Bak were procured from Upstate (Lake Placid, N.Y.). PARP (85) was obtained from Promega (Madison, Wis.). Anti-mouse and anti-rabbit secondary antibody horseradish peroxidase conjugate was obtained from Amersham Life Science Inc. (Arlington Height, Ill.). Akt-siRNA and scrambled-siRNA were purchased from Dharmacon (Lafayette, Colo.). Fisetin was purchased from Sigma Chemical Co. (St. Louis, Mo.). BCA Protein assay kit was obtained from Pierce (Rockford, Ill.). Novex precast Tris-glycine gels were obtained from Invitrogen (Carlsbad, Calif.). The Apo-Direct kit for flow cytometry was purchased from Phoenix Flow Systems (San Diego, Calif.). Magic Red™ caspase detection kit was purchased from Immunochemistry Technologies, LLC (Bloomington, Minn.). Annexin-V-Fluos staining kit and Cell Death Detection ELISAPLUS kit was from Roche Diagnostics Corporation (Indianapolis). ApoAlert Cell Fractionation kit was purchased from BD Biosciences Clontech (Palo Alto, Calif.).

Cell culture and treatment. The LNCaP, CWR22Ru1 and PC-3 cells were

obtained from ATCC (Manassas, Va.). LNCaP cells were cultured in Dulbecco's Modified Eagle's medium supplemented with 5% heat-inactivated fetal bovine serum and 1% penicillin/streptomycin (P-S). CWR22Ru1 and PC-3 cells were grown in RPMI 1640 (Invitrogen) supplemented with 10% fetal bovine serum and 1% P-S. Human prostate epithelial cells (PrEC) were obtained from Cambrex Bioscience (Walkersville, Md.) and grown in prostate epithelial basal cell medium (Cambrex Bioscience) according to the manufacturer's instructions. The cells were maintained under standard cell culture conditions at 37° C. and 5% CO₂ in a humid environment. Fisetin dissolved in dimethyl sulfoxide (final concentration 0.1% v/v) was used for the treatment of cells. The cells (60-70% confluent) were treated with fisetin (10-60 μM) for 24 and 48 h in complete growth medium.

Cell viability. The effect of fisetin on the viability of cells was determined by 3-[4,5-dimethylthiazol-2-yl]-2,5-diphenyl tetrazoliumbromide assay. LNCaP, CWR22Ru1, PC-3 and PrEC cells were plated at 1×10⁴ cells per well in 200 μl of complete culture medium containing 10-60 μM concentrations of fisetin in 96-well microtiter plates for 24 and 48 h. After incubation for specified times at 37° C. in a humidified incubator, 3-[4,5-dimethylthiazol-2-yl]-2,5-diphenyl tetrazoliumbromide (5 mg/ml in PBS) was added to each well and incubated for 2 h, after which the plate was centrifuged at 1,800×g for 5 min at 4° C. The absorbance was recorded on a microplate reader at the wavelength of 540 nm. The effect of fisetin on growth inhibition was assessed as percent cell viability where DMSO-treated cells were taken as 100% viable. DMSO at the concentrations used was without any effect on cell viability.

DNA Cell Cycle Analysis. The LNCaP cells (50-60% confluent) were treated with fisetin (10-60 μM; 48 h) in complete medium. The cells were trypsinized thereafter, washed twice with cold PBS and centrifuged. The cell pellet was resuspended in 50 μl cold PBS to which cold methanol (450 μL) was added and the cells were incubated for 1 h at 4° C. The cells were centrifuged at 1,100 rpm for 5 minutes, pellet washed twice with cold PBS, suspended in 500 μl PBS and incubated with 5 μl RNase (20 μg/ml final concentration) for 30 min. The cells were chilled over ice for 10 min and incubated with propidium iodide (50 μg/ml final concentration) for 1 h for analysis by flow cytometry. Flow cytometry was performed with a FACScan (Becton Dickinson). A minimum of 10,000 cells per sample were collected and the DNA histograms were further analyzed by using ModiFitLT software (Verily Software House, Topsham, Me.) for cell cycle analysis.

Protein extraction and western blotting. Following the treatment of LNCaP cells with fisetin (10-60 μM; 48 h), the media was aspirated, the cells were washed with cold PBS (pH 7.4), and ice-cold lysis buffer (50 mM Tris-HCl, 150 mM NaCl, 1 mM EGTA, 1 mM EDTA, 20 mM NaF, 100 mM Na₃VO₄, 0.5% NP-40, 1% Triton X-100, 1 mM PMSF (pH 7.4)) with freshly added protease inhibitor cocktail (Protease Inhibitor Cocktail Set III, Calbiochem, La Jolla, Calif.) over ice for 30 min. The cells were scraped and the lysate was collected in a microfuge tube and passed through a needle to break up the cell aggregates. The lysate was cleared by centrifugal ion al 14,000 g for 15 min at 4° C. and the supernatant (whole cell lysate) was used or immediately stored at −8020 C.

For western blotting, 30-50 μg protein was resolved over 8-12% polyacrylamide gels and transferred to a nitrocellulose membrane. The blot was blocked in blocking buffer (5% nonfat dry milk/1% Tween 20; in 20 mM TBS, pH 7.6) for 1 hr at room temperature, incubated with appropriate monoclonal or polyclonal primary antibody in blocking buffer for 1.5 h to overnight at 4° C., followed by incubation with anti-mouse or anti-rabbit secondary antibody horseradish peroxidase conjugate obtained from Amersham Life Science Inc. and detected by chemiluminescence and autoradiography using XAR-5 film obtained from Eastman Kodak Co. (Rochester, N.Y., USA). Densitometric measurements of the band in Western blot analysis were performed using digitalized scientific software program UN-SCAN-IT (Silk Scientific Corporation. Orem, Utah.

Apoptosis assessment by annexin-V staining. The Annexin-V-Fluos staining kit was used for the detection of apoptotic cells according to vendor's protocol. This kit uses a staining protocol in which the apoptotic cells are stained with annexin-V (green fluorescence). The LNCaP cells were grown to 70% confluency and treated with fisetin (10-60 μM) for 48 h. The fluorescence was measured by a Zeiss 410 confocal microscope (Thornwood, N.Y.). Confocal images of green annexin-FITC fluorescence were collected using 488 nm excitation light from an argon/krypton laser, a 560 nm dichroic mirror and a 514-540 nm bandpass barrier filter.

Apoptosis by ELISA. Following treatment of LNCaP cells with fisetin (10-60 μM; 48 h), the extent of apoptosis was determined by Cell Death Detection ELISA^{PLUS} assay, according to the manufacturer's protocol. The whole cell lysate (40 μg of total protein) was added to the lysis buffer and pipetted on a streptavidin-coated 96-well microtiter plate to which the immunoreagent mix was added and incubated for 2 h at room temperature, with continuous shaking at 500 rpm. The wells were then washed with washing buffer, the substrate solution added, the color developed (10-20 min) was read at 405 nm against the blank, reference wavelength of 490 nm. The enrichment factor (total amount of apoptosis) was calculated by dividing the absorbance of the sample (A405 nm) by the absorbance of the controls without treatment (A490 nm).

Immunofluorescence staining for active caspase-3 and -7. LNCaP cells were incubated with 40 μM concentration of the general caspase inhibitor Z-VAD-FMK for 2 h, followed by the addition of fisetin (40 μM; 48 h) and then harvested. ICT's Magic Red™ substrate-based MR-Caspase assay kit was used for the immunofluorescence staining for caspase-3 and -7. This kit utilizes the fluorophore, cresyl violet. As the 4 amino acid sequence, aspartylglutamylalanylaspartic acid (DEVD) is the optimal sequence for caspase -3 as well as -7, it was coupled to cresyl violet to create the caspase 3/7 substrate, MR-(DEVD)2. When bi-substituted via amide linkage to two target caspase sequence groups {(DEVD)2}, cresyl violet does not fluoresce. Following enzymatic hydrolysis at one or both of the aspartic acid amide linkage sites, the mono and non-substituted cresyl violet fluorophores fluoresce red and fluorescence photographs were obtained at 510-560 nm excitation and 610 nm emission.

Detection of cytochrome c release. After the treatment of LNCaP cells with fisetin (10-60 μM; 48 h), the mitochondrial and cytosolic fractions were prepared according to manufacturer's instructions. After the separation of cytosolic and mitochondrial fraction, 40 μg protein was resolved over 12% polyacrylamide gels and immunoblotted with the cytochrome c and Cox-4 antibodies.

Silencing of Akt by Small Interfering RNA. LNCaP cells were transfected with Akt-siRNA (75 nmol) and scrambled siRNA (75 nmol) procured from Dharmacon (Lafayette, Colo.) using the Nucleofection Kit R specific for LNCaP transfection from Amaxa Biosystems (Gaithersburg, Md.). Cells were resuspended in a solution from nucleofector kit R following the manufacturer's guidelines. 100 μl of nucleofector solution R was mixed with 2×10⁶ cells and siRNA. They were then transferred to the cuvette provided with the kit and were nucleofected with an Amaxa Nucleofector apparatus. Cells were transfected using the T-001 pulsing parameter and were transferred into 100-mm plates containing 37° C. prewarmed culture medium. After transfection, cells were cultured and the medium was replaced with fresh medium. Cells were treated with 40 μM fisetin for 48 h, and protein lysates were prepared. Using 2 μg of GFP we observed 70-80% transfection efficiency with this protocol.

Statistical Analysis. Results were analyzed using a two-tailed Student's t-test to assess statistical significance and P values <0.05 were considered significant.

Results. Inhibition of cell growth by fisetin in LNCaP cells but not in PrEC cells. The dose- and time-dependent effect of treatment with fisetin (10-60 μM) on the growth of human prostate cancer (LNCaP, CWR22Ru1, PC-3) cells and normal prostate epithelial (PrEC) cells was investigated. The effect of fisetin on the growth of these cells by 3-[4,5-dimethylthiazol-2-yl]-2,5-diphenyl tetrazoliumbromide (MTT) assay was evaluated. The antiproliferative effects of fisetin on LNCaP, CWR22Ru1, PC-3 and PrEC cells was compared.

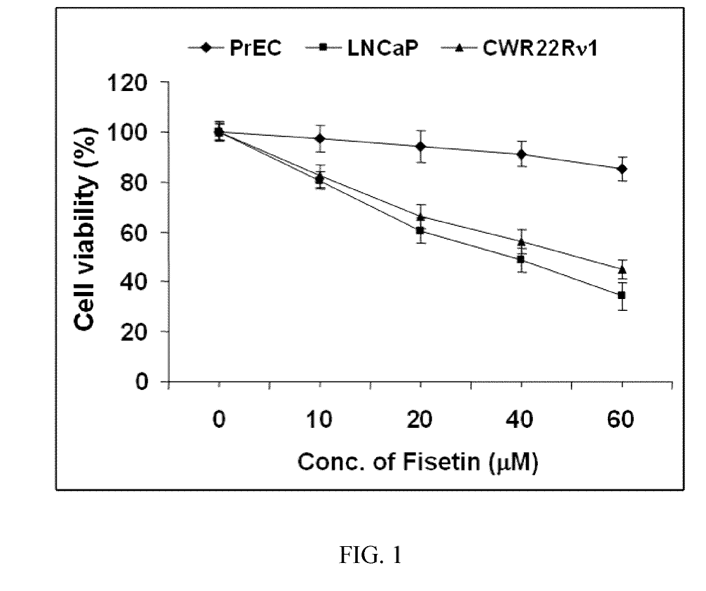

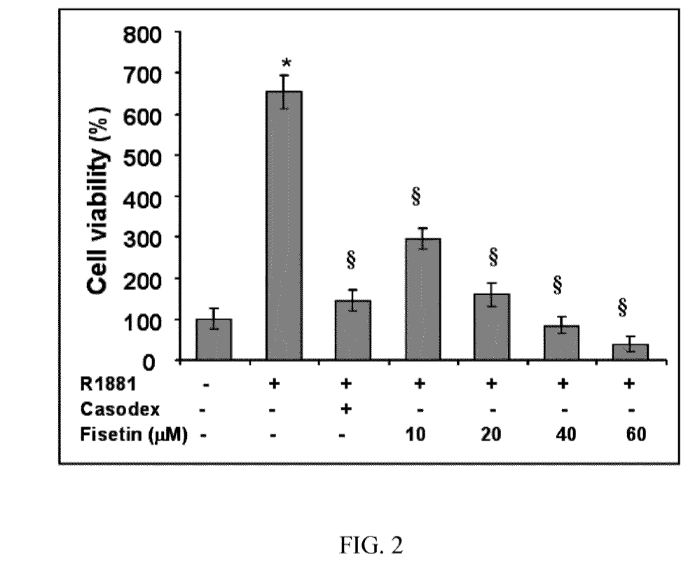

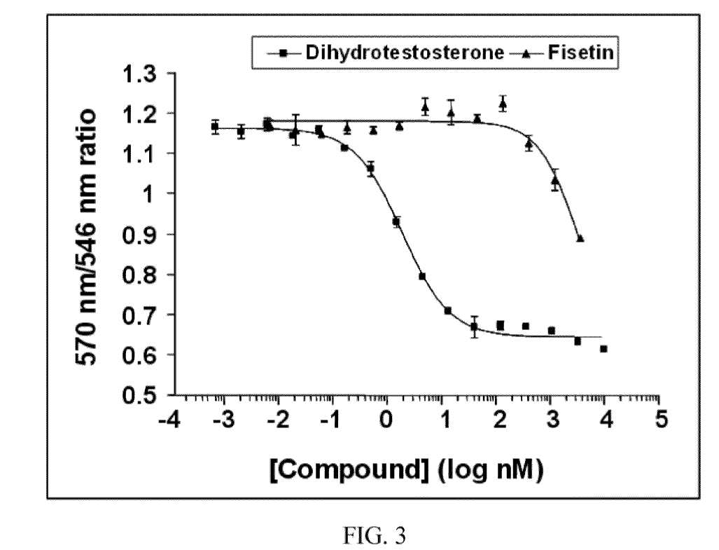

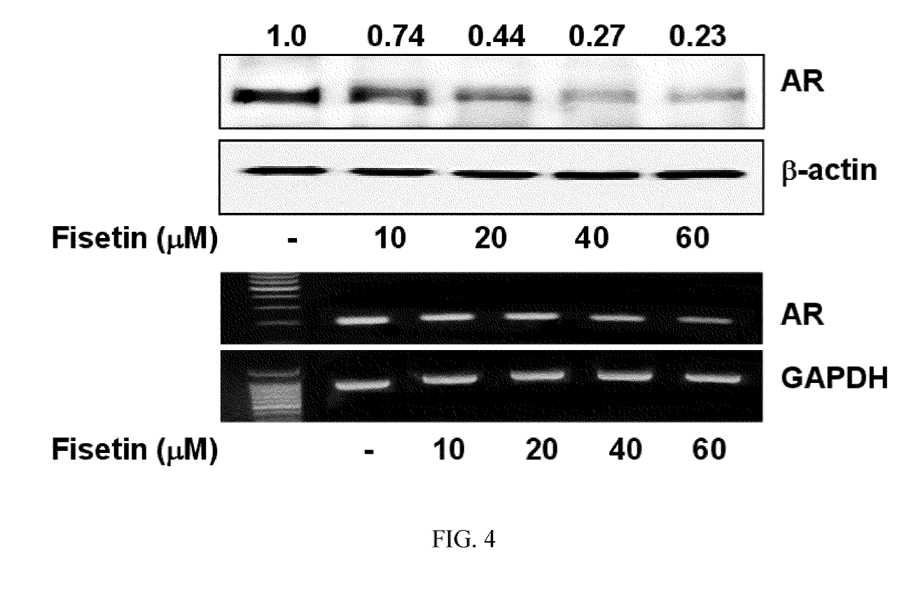

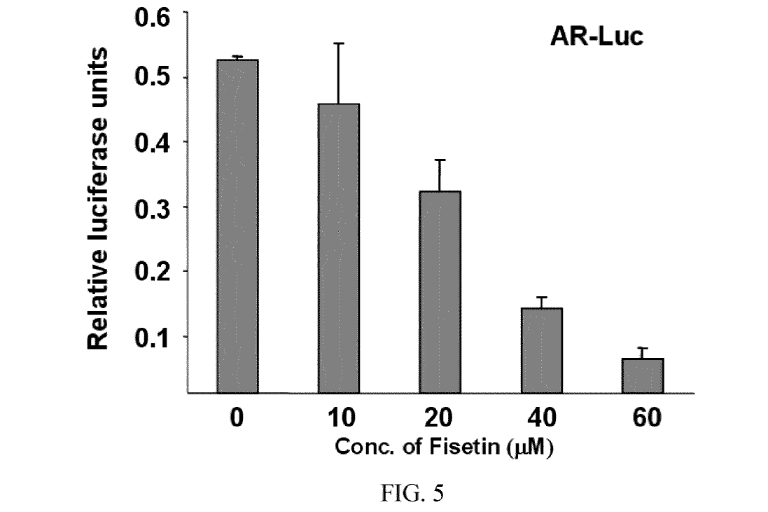

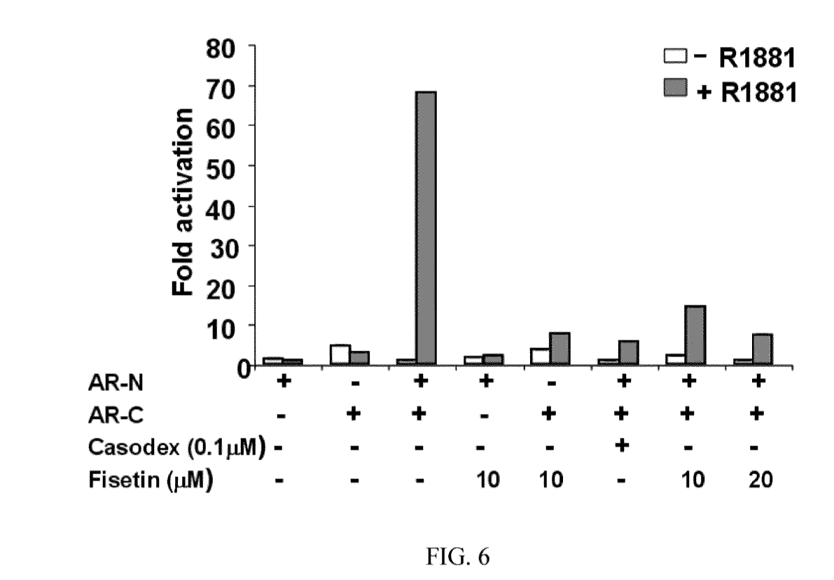

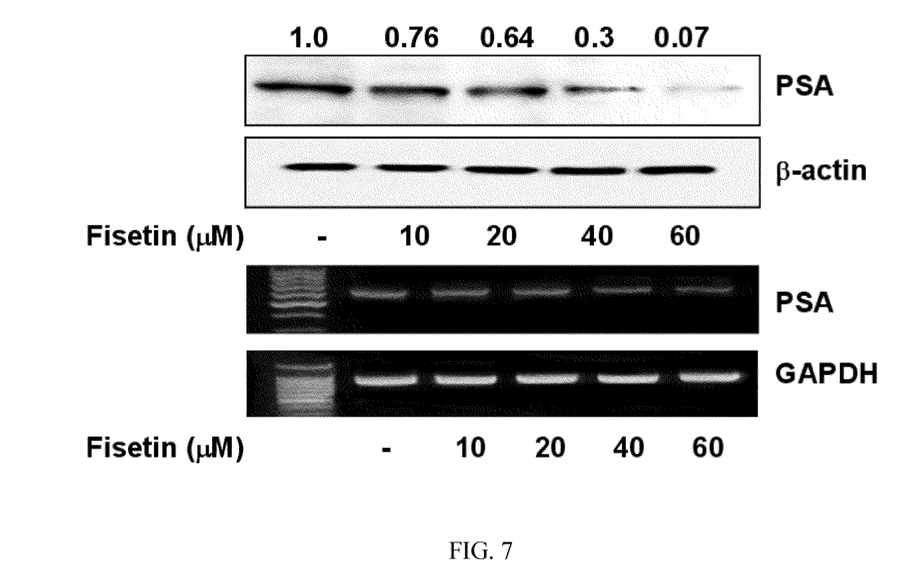

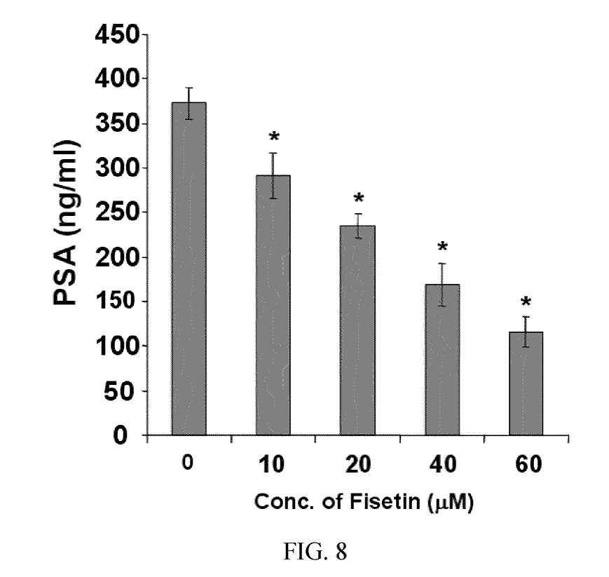

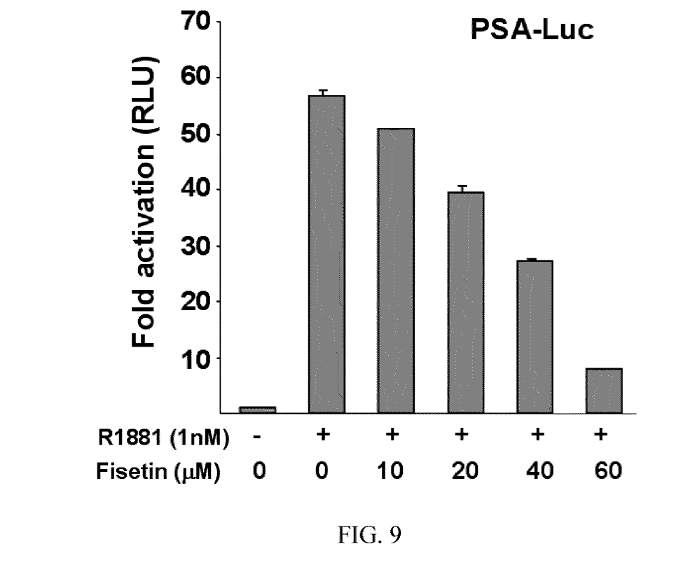

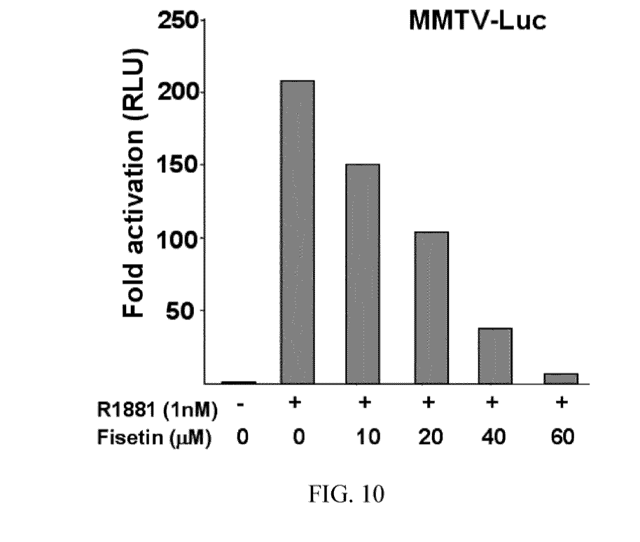

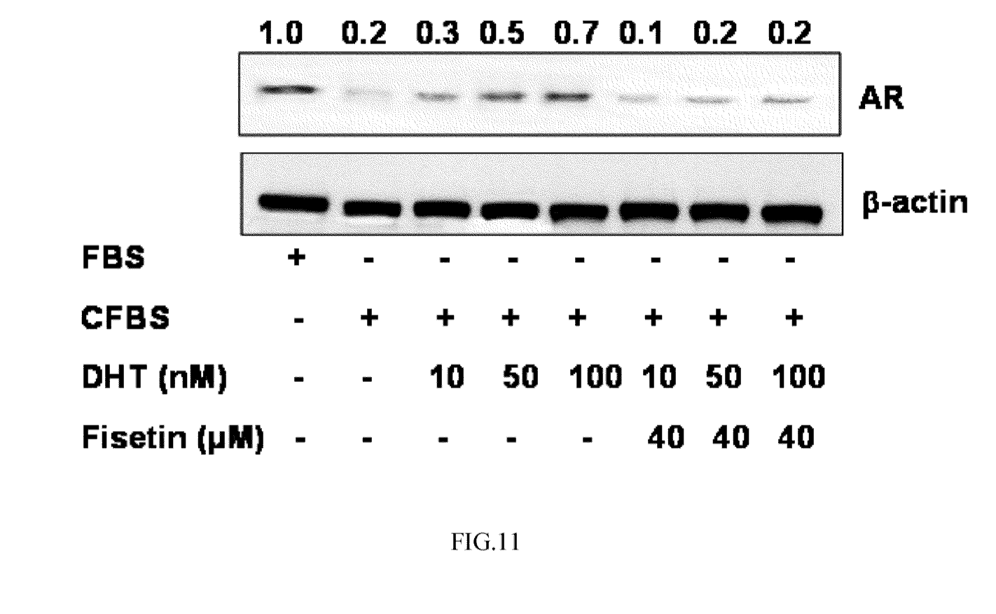

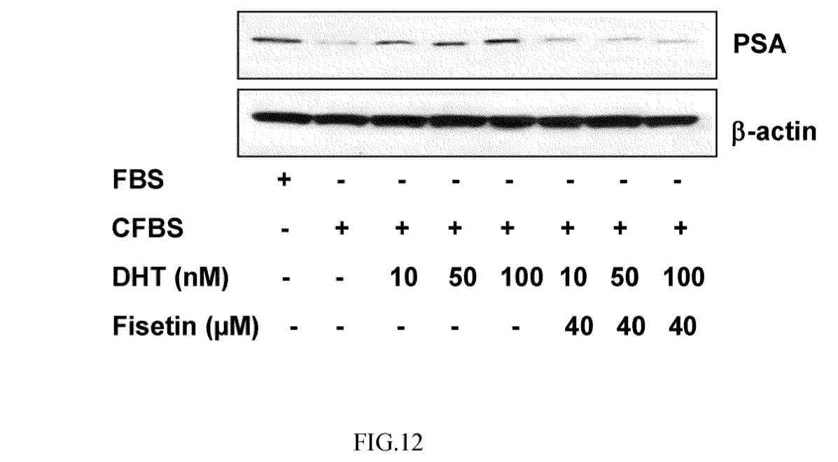

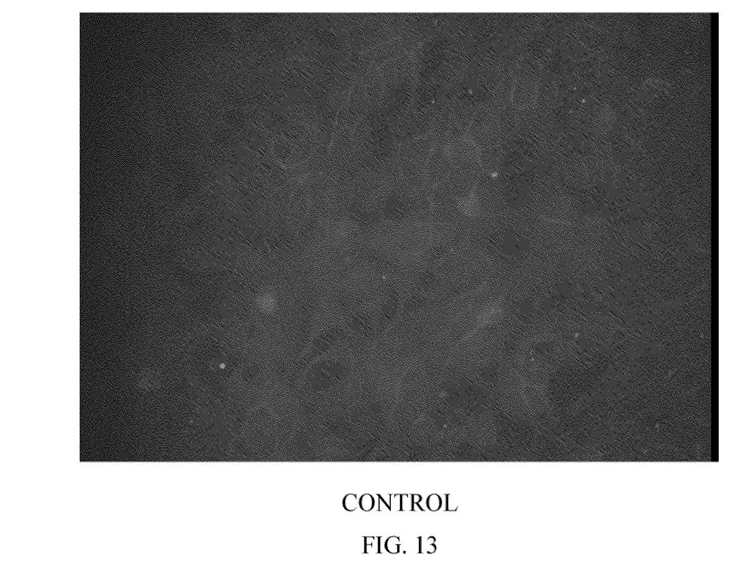

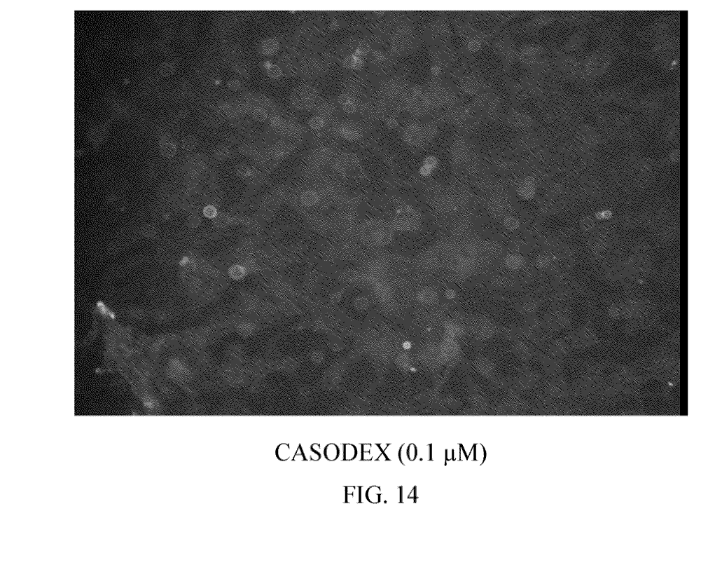

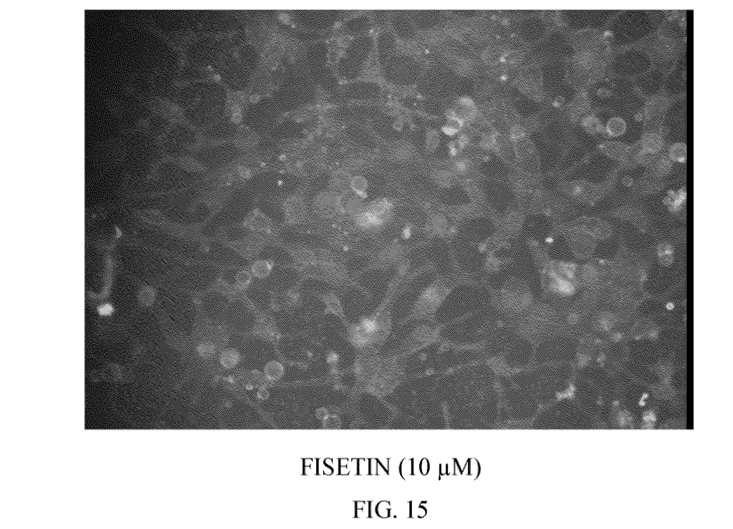

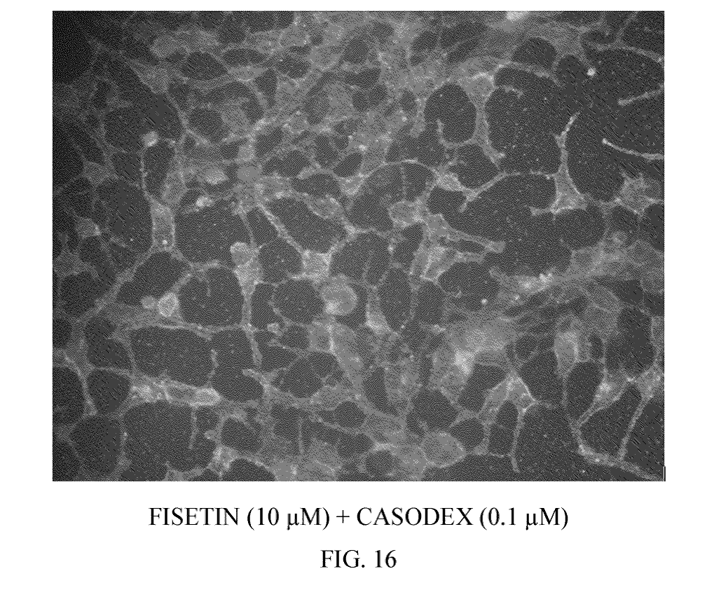

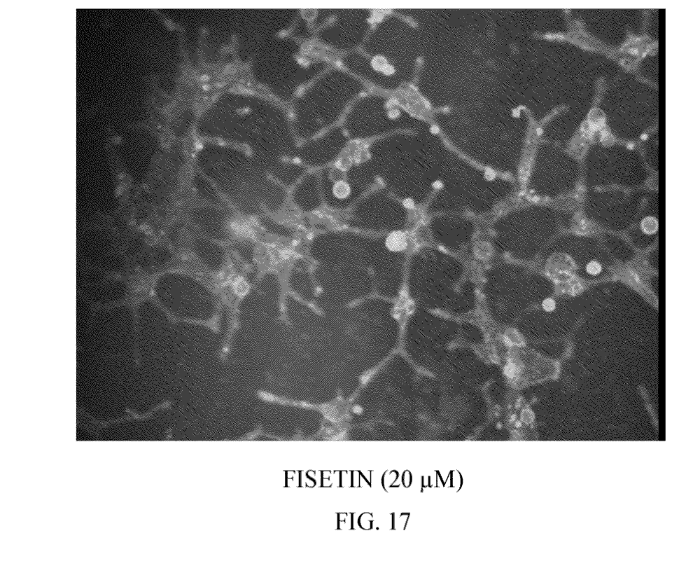

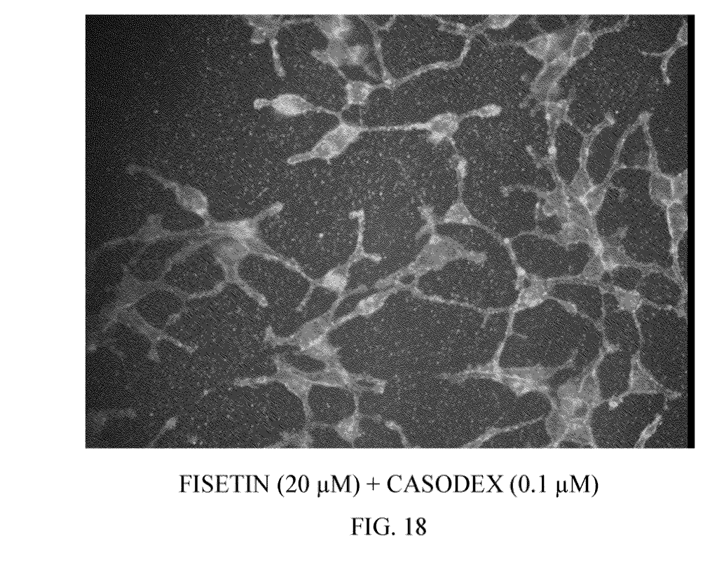

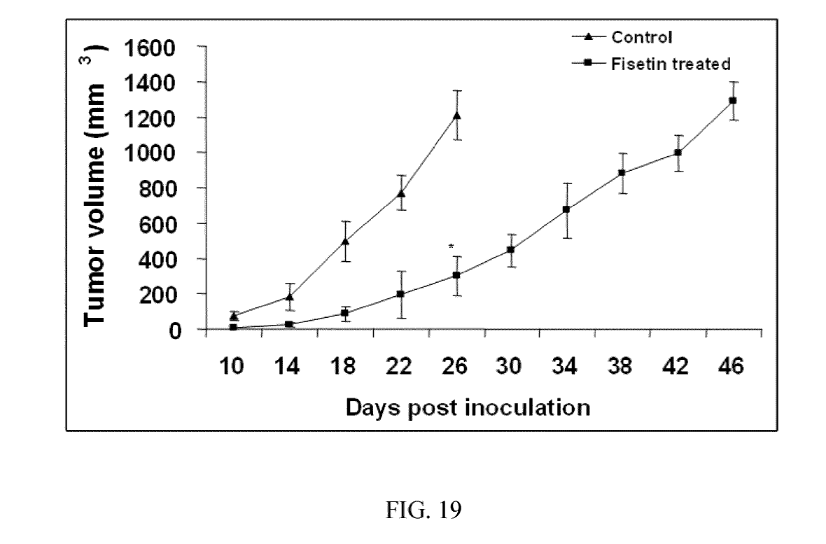

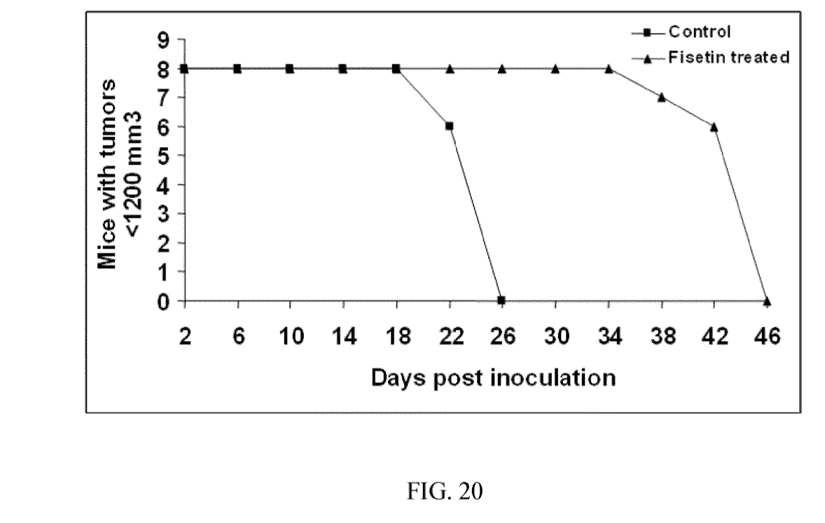

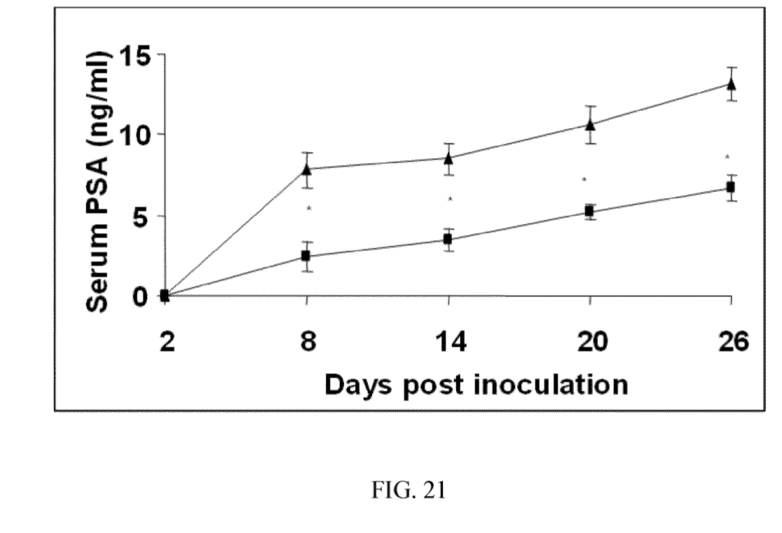

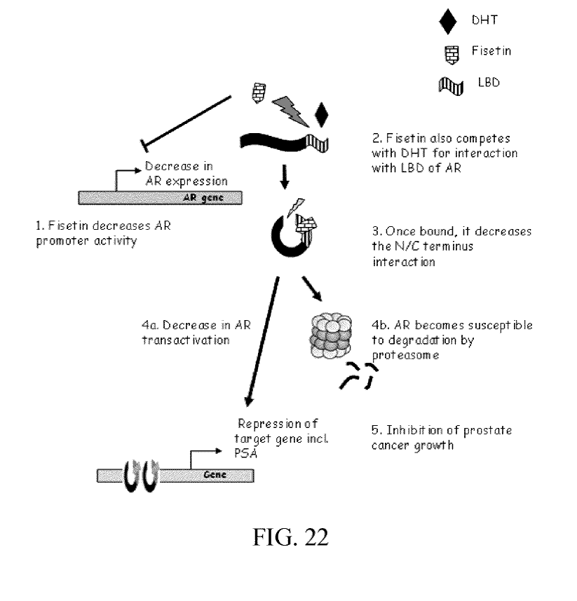

As shown in FIGS. 1, 23 and 24, Treatment with fisetin (10-60 μM) for 24 h decreased cell viability in LNCaP (15, 33, 45 and 57%), CWR22Ru1 (12, 26, 38 and 52%) and PC-3 (7, 18, 24 and 33%) cells but had minimal effect on PrEC cells at these doses. At 48 h, there was a much pronounced decrease in cell viability upon treatment with fisetin (10-60 μM) in LNCaP (19, 40, 49 and 62%), CWR22Ru1 (18, 34, 44 and 55%) and PC-3 (11, 25, 32 and 46%) cells. But, as shown in FIG. 24, fisetin had minimal effect on PrEC cells. As demonstrated by the comparative data, fisetin treatment caused a synergistic and maximum decrease in cell-viability in LNCaP cells as compared to CWR22Ru1 and PC-3 cells.

G1 phase cell cycle arrest by fisetin in LNCaP cells. Fisetin-induced growth inhibition of the cells being mediated via alterations in cell cycle was evaluated. The effect of fisetin on cell cycle distribution was also evaluated. DNA cell cycle analysis using LNCaP cells and fisetin treatment was found to result in a significant dose-dependent increase of cell population in the G1 phase of the cell cycle. As shown in FIGS. 25-29, the G1 phase cell cycle distribution was 61, 63, 67 and 69% at 10, 20, 40 and 60 μM fisetin concentrations, respectively. The increase in the G0/G1 phase cell population was accompanied by a concomitant decrease in the S phase and G2/M phase cell populations.

Inhibition of cyclins, cdks and induction of WAF1/p21, KIP1/p27 by fisetin in LNCaP cells. The protein expression of the cyclins and cdks are operative in G1 phase of the cell cycle. As shown in FIG. 30, fisetin treatment of LNCaP cells caused a dose-dependent decrease in the protein expression of cyclins D1, D2 and E. The decrease in cyclin D2 protein expression was more pronounced than that of cyclin D1 and cyclin E. Using immunoblot analysis, as shown in FIG. 31, treatment of LNCaP cells with fisetin resulted in a dose-dependent decrease in cdk2, 4 and 6.

The effect of fisetin treatment on the induction of WAF1/p21 (which is known to regulate the entry of cells at G1 to S transition checkpoint) was evaluated. As shown in FIG. 32, immunoblot analysis and relative density of the bands revealed that treatment with fisetin resulted in induction of WAF1/p21 and KIP1/p27 even at the lowest concentration of 10 μM with a significant increase at the highest concentration of 60 μM.

Induction of apoptosis by fisetin in LNCaP cells. Annexin-V staining was performed to determine apoptotic cells following fisetin treatment to LNCaP cells. Annexin-V specially binds to phosphatidylserine and has been employed to determine apoptotic cells.

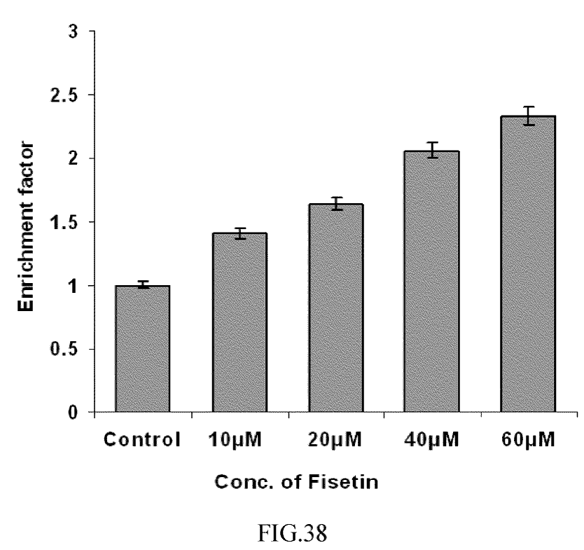

As shown in FIGS. 33-37, when LNCaP cells were stained with annexin-V and examined under a fluorescence microscope, apoptotic cells were found to be increased in fisetin (10-60 μM; 48 h) treated cells. As shown in FIG. 38, treatment with fisetin resulted in significant apoptosis in LNCaP cells (40, 64, 103 and 106% al 10, 20, 40 and 60 μM concentrations of fisetin, respectively, 48 h), as compared to control in a dose-dependent fashion. As shown in FIG. 39-41, PARP cleavage analysis showed that the full size PARP (116 KD) protein was cleaved to yield an 85 KD fragment after treatment of cells with fisetin (as shown by immunoblot analysis and relative density of the bands). We also performed ELISA to evaluate the induction of apoptosis by fisetin.

Modulation of Bcl2-family proteins by fisetin in LNCaP cells. Bax and Bcl-2 play significant role in apoptosis. The effect of treatment with fisetin on the constitutive protein levels of Bax and Bcl-2 in LNCaP cells was evaluated. As shown in FIG. 42, the immunoblot analysis and relative density of the bands exhibited a significant increase in the protein expression of Bax at 10-60 μM concentration of fisetin. In sharp contrast, the protein expression of Bcl-2 was significantly decreased by fisetin treatment in a dose-dependent fashion.

As shown in FIG. 43, a significant dose-dependent shift in the ratio of Bax to Bcl-2 was observed after treatment with fisetin indicating the induction of apoptotic process. The protein levels of Bak, Bad, Bid (proapoptotic) and Bcl-xL (antiapoptotic) was also evaluated. As shown in FIG. 44, the immunoblots revealed a significant increase in the protein expression of Bak, Bad and Bid. The immunoblot further showed a significant decrease in Bcl-xL expression by treatment with fisetin in a dose-dependent fashion in LNCaP cells further confirming the induction of apoptotic process.

Induction of mitochondrial release of cytochrome c into cytosol by fisetin in LNCaP cells. The release of cytochrome c (one of the most important respiratory-chain proteins located in the mitochondria) into the cytosol is the hallmark of apoptosis of cells treated with certain apoptosis inducers. (Khan N et al., 2007,Apoptosis by dietary factors: the suicide solution for delaying cancer growth, Carcinogenesis 28:233-239). The cylosolic cytochrome c was measured using immunoblotting in LNCaP cells that were treated for 48 h with 10-60 μM of fisetin.

As shown in FIG. 45, the levels of cytochrome c in the cytosol were elevated dose-dependently after treatment with fisetin. The observed cytoplasmic cytochrome c was verified as not due to mechanical disruption of the mitochondria. Simultaneous analysis was performed for cytochrome c oxidase (Cox-4), which was not detected in the cytosolic extracts of any samples.

Inhibition of XIAP and induction of Smac/DIABLO by fisetin in LNCaP cells. Dysregulation of apoptosis has an important role in cancer and resistance to chemotherapy. The XIAP has been considered to be the most potent caspase inhibitor of all known IAP family members. Recently, an antagonist of XIAP has been identified, which is referred to as Smac/DIABLO. (Huang Y et al., 2001, Structural basis of caspase inhibition by XIAP: differential roles of the linker versus the BIR domain, Cell 104:781-790).

The relevance of antiapoptotic XIAP and proapoptotic Smac/DIABLO in prostate cancer was evaluated. XIAP and Smac/DIABLO protein expression in LNCaP cells were analyzed after treatment with fisetin. As shown in FIG. 46, using immunoblot analysis and relative density of the bands, treatment with fisetin (10-60 μM; 48 h) caused inhibition of XIAP and induction of Smac/DIABLO in LNCaP cells.

Induction of active caspase-3,-8 and -9 by fisetin in LNCaP cells. The apoptotic program was executed by a family of highly conserved cysteinyl aspartate-specific proteases known as caspases, which dismantle the cell in an orderly fashion by cleaving a large number of cellular substrates. Modulating the mechanisms of caspase activation and suppression is a critical molecular target in chemoprevention because these processes lead to apoptosis. As shown by immunoblot analysis and relative density of the bands in FIG. 47, treatment of cells with fisetin resulted in a dose-dependent increase in the activation of casapse-3, -8 and -9.

Inhibition of fisetin-induced induction of caspases by Z-VAD-FMK in LNCaP cells. These results indicated that fisetin treatment of LNCaP cells resulted in apoptosis via activation of caspases. Z-VAD-FMK inhibits caspase activation. Z-VAD-FMK blocking of fisetin-mediated apoptosis was evaluated. As shown in FIG. 48, compared to fisetin treatment (10-60 μM), pre-incubation of LNCaP cells with Z-VAD-FMK (40 μM) for 2 h before fisetin treatment resulted in significant decrease in the protein expression of active caspase-3, -8 and -9 as observed by immunoblot analysis.

Active caspase -3 & -7 activity was also detected by immunofluorescence staining within whole living LNCaP cells using ICT's Magic Red™ substrate-based MR-Caspase assay kit. The MR-Caspase photostable fluorogenic substrate easily penetrates the cell membrane and the membranes of the internal cellular organelles, entering the cell in the non-fluorescent state. In the presence of caspase-3 and -7 enzymes (DEVDases), the 4 amino acid (DEVD) caspase target sequences are cleaved off yielding a red fluorescent product. DEVDase mediated production of the red fluorophore signals apoptotic activity within that particular cell.

As shown in FIGS. 49-52, upon treatment with Z-VAD-FMK (40 μM) for 2 h before fisetin treatment (40 μM), there was significant decrease in the immunofluorescence staining of active caspase-3 & -7. Similarly, when cells were treated with fisetin only (40 μM), there was intense staining of active caspase-3 & 7 as detected by red fluorescent product.

Inhibition of PI3K and phosphorylation of Akt protein expression by fisetin in LNCaP cells. The PI3K/Akt signaling pathway and its downstream transcription factors have been studied in detail for their role in cell proliferation, survival, cycle control, as well as other cellular functions. Accumulating evidence demonstrates that dysregulation of this pathway also plays an essential role in cancer development. The effect of fisetin on PI3K protein expression in LNCaP human prostate carcinoma cells was evaluated.

As shown in FIG. 53, western blot analysis revealed that fisetin (10-60 μM) caused inhibition in the expression of regulatory (p85) subunit of PI3K in LNCaP cells. Treatment of LNCaP cells with fisetin (10-60 μM) also inhibited phosphorylation of Akt at both Ser473 and Thr308.

Akt-dependent modulation in the Bcl2 family proteins by fisetin in LNCaP cells. To assess whether fisetin-induced apoptosis was Akt-dependent, Akt was silenced using small interfering RNA (siRNA). Silencing of Akt by siRNA caused an increase in the protein expression of proapoptotic Bad and Bax. As shown in FIG. 54, it also caused a decrease in the protein expression of Bcl2 and Bcl-xL. Fisetin treatment (40 μM) to Akt-siRNA treated cells also augmented the increase in Bad and Bax and decrease in Bcl2 and Bcl-xL suggesting that these effects are mediated in part through Akt.

Fisetin binds to the AR: in silico evidence.

Androgens bind to the AR. Ligands that block the actions of androgens are used clinically for the treatment of prostate cancer. Such drugs include a steroidal antiandrogen cyproterone acetate (Cyprostat™) and non-steroidal antiandrogens flutamide (Eulexin™) and bicalutamide (Casodex™).

In patients treated with such drugs, mutations in patient's AR causes drug

resistance, which increases the drug's agonist activity. Discontinuation of drug therapy may also cause antiandrogen withdrawal syndrome. In regions where ligands contact directly with ARs, a number of antiandrogen withdrawal syndrome mutations occur. Such mutations compensate for the increased antagonist bulkiness as compared to the effects of agonist compounds.

Due to such mutations, antagonist ligands cause the same AR ligand binding domain (AR-LBD) conformation as the agonist-bound AR-LBD. Molecular modeling is used to analyze the potential for a docking solution, which is useful to understand how drugs (more bulky than steroidal agonists) can be accommodated within the AR.

In addition to potent natural androgens, the AR binds to a variety of synthetic agonist or antagonist molecules having different affinities. Fisetin acts as an AR ligand by competing with high-affinity androgen to interact with the ligand binding domain of AR. Thus, fisetin physically binds to the AR, which is important for imparting anti-androgenic effects.

The in silico approach was used to identify molecular determinants responsible for such selectivity using AutoDock4 for molecular docking analysis. AutoDock4 was used to determine that fisetin binds the AR, which has a known 3D structure.

Fisetin was docked using 2PNU.pdb as the starting receptor docking followed by molecular modeling using the SYBYL® (Tripos. Corp, St. Louis) program. SYBYL® is a general molecular modeling program that is also a comprehensive computational tool kit used for molecular design and analysis. It also has a special focus on the creation of new chemical entities. SYBYL® provides useful construction and analysis tools for both organic and inorganic molecular structures.

AutoDock4 scoring was done based upon an estimated free energy of binding that includes a summation of the final intermolecular docking energy, total internal energy, and, ligand torsional free energy—minus system unbound energy. (Morris et al., 1998, Automated docking using a Lamarckian genetic algorithm and empirical binding free energy function, J. Computational Chemistry 19:1639-1662).

Fisetin docked with the AR with a binding free energy of E=−7.46 Kcal/mole ˜3.4 μM. Fisetin also formed hydrogen bonds with the Arg752 sidechain nitrogen, the Asn705 sidechain nitrogen, and, the Leu873 backbone oxygen. (See FIG. 55).

Hypothetical model of the AR in complex with fisetin is shown in FIG. 55. Panel A: Molecular modeling studies using AutoDock and 2PNU.pdb as the starting receptor docking site showed that fisetin binds to the AR. Domains of the AR are presented in various shades. (See inset). Fisetin bound to the receptor. Panel B: Inset enlarged with putative binding sites. The hydrogen bonds are depicted as dashed lines: N705 (asparagine705); L873 (leucine873): R752 (arginine752). In silico molecular modeling analysis also determined that fisetin docks with the AR with E=−7.46 Kcal/mole ˜3.4 μM. Fisetin forms hydrogen bonds with the Arg752 sidechain nitrogen, the Asn705 sidechain nitrogen, and the Leu873 backbone oxygen.

We claim:

1. A method of treating androgen dependent prostate cancer in a human comprising administering a therapeutically effective dose of an active pharmaceutical ingredient comprising fisetin according to the formula 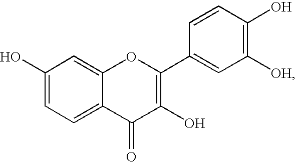
or, a prodrug, salt, hydrate, solvate or solute thereof.

2. A method of treating androgen dependent prostate cancer in a human comprising orally administering a therapeutically effective dose of an active pharmaceutical ingredient comprising fisetin according to the formula 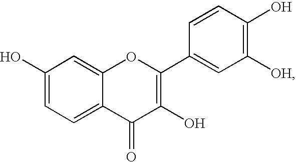
or, a prodrug, salt, hydrate, solvate or solute thereof.

3. A method of treating androgen dependent prostate cancer in a human comprising transdermally administering a therapeutically effective dose of an active pharmaceutical ingredient comprising fisetin according to the formula 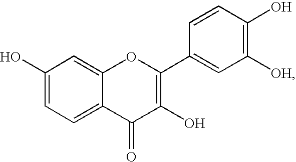
or, a prodrug, salt, hydrate, solvate or solute thereof.

4. A method of treating androgen dependent prostate cancer in a human comprising topically administering a therapeutically effective dose of an active pharmaceutical ingredient comprising fisetin according to the formula 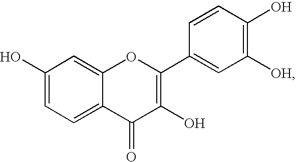
or, a prodrug, salt, hydrate, solvate or solute thereof.

5. The method of any one of claims 1-4, wherein the therapeutically effective dose is around 50 mg/kg_{BW}.
